SEQUENCE LISTING (2 sequences)

SEQ ID NO: 1 - DNA, 1741 bp, Homo sapiens
ctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccattcctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaaccctcggccagcaggtcccctatgccaccaagggcaaccagtgggtaggatacgacgaccaggaaagcgtcaaaagcaaggtgcagtacctgaaggataggcagctggcaggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatcctacttccccttcctaattccacagctgctcaataaagtacaagagtttaacagtgtg

SEQ ID NO: 2 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRTLGQQVPYATKGNQWVGYDDQESVKSKVQYLKDRQLAGAMVWALDLDDFQGSFCGQDLRFPLTNAIKDALAAT

(19) United States
(12) Patent Application Publication
(10) Pub. No.: US 2011/0070601 A1
(43) Pub. Date: Mar. 24, 2011

(54) CLASSIFICATION OF INDIVIDUALS SUFFERING FROM CARDIOVASCULAR DISEASES ACCORDING TO SURVIVAL PROGNOSES AS FOUND BY MEASURING THE LEVELS OF BIOMARKER YKL-40

(75) Inventor: Jens Kastrup, Birkerød (DK)

(73) Assignee: Rigshospitalet, Copenhagen Ø (DK)

(21) Appl. No.: 12/863,748

(22) PCT Filed: Jan. 22, 2009
(86) PCT No.: PCT/DK2009/050015

§ 371 Date: Oct. 26, 2010

(30) Foreign Application Priority Data
Jan. 23, 2008 (DK) PA 2008 00089

(51) Int. Cl. C12Q 1/34; C12Q 1/44; C12M 1/34; C12Q 1/37

(52) U.S. Cl. 435/19; 435/18; 435/287.1; 435/23

(57) ABSTRACT

The present invention relates to the method of measuring the YKL-40 level and using this measurement as a prognosis for survival of an individual suffering from heart disease caused by atherosclerosis. The method may be used for classification of individuals in order to optimize treatment or monitoring the individuals during the course of or prior to or after treatment. The individual may suffer from any type of cardiovascular disease or disorder. The method also detects and determines whether diagnostically or prognostically significant levels of YKL-40 molecules are present in a biological sample. Furthermore the level of YKL-40 may be used to predict disease relapse.

All patent and non-patent references cited in the application, or in the present application, are also hereby incorporated by reference in their entirety.

### FIELD OF INVENTION

The present invention relates to the method of detecting the biomarker YKL-40 as a prognostic marker of survival of an individual. The individual may suffer from any type of cardiovascular disease or disorder and the found level of YKL-40 enables classification and/or monitoring of the individual according to survival prognosis.

### BACKGROUND OF INVENTION

Cardiovascular disease is the number one cause of death globally and is projected to remain the leading cause of death according to the World Health Organization. An estimated 17.5 million people died from cardiovascular disease in 2005, representing 30% of all global deaths. Of these deaths, 7.6 million were due to heart attacks and 5.7 million were due to stroke. If appropriate action is not taken, by 2015, an estimated 20 million people will die from cardiovascular disease every year, mainly from heart attacks and strokes.

Cardiovascular diseases are caused by disorders of the heart and blood vessels, and include coronary heart disease (heart attacks), coronary artery disease, raised blood pressure (hypertension), peripheral artery disease, rheumatic heart disease, congenital heart disease and heart failure among others. By the time the cardiovascular disorders are detected, the underlying cause, which most often is atherosclerosis, may be advanced, having progressed for decades. The major causes of cardiovascular disease are tobacco use, physical inactivity, and an unhealthy diet.

Treatment of cardiovascular disease depends on the specific form of the disease in each patient. Medications, such as anti-angina and blood pressure reducing medications, aspirin and statin cholesterol-lowering drugs may be helpful. Often, surgery or angioplasty may be warranted to reopen, repair, or replace damaged blood vessels, such as in bypass operations, percutaneous coronary interventions, and the installment of pacemakers, and as a last resort warrant heart transplantation.

Administering the best possible treatment for each individual patient would improve the efficacy of any treatment whether it involves administration of medicaments, surgery, or other and independent of whether the treatment given is curative or ameliorative. A classification of the individuals suffering from cardiovascular disease or disorders according to survival prognosis would be of assistance in choosing the best possible treatment, improve the effect of an administered treatment, improve the survival rate, lower relapse risks, and heighten the quality of life following the occurrence of a cardiovascular disease. Furthermore, the ability to monitor this group of individuals would be of assistance in choosing the most effective immediate and follow-up treatment, and be of guidance when counseling on lifestyle chances required subsequent to the occurrence of a cardiovascular disease or disorder.

### SUMMARY OF INVENTION

The present invention as described herein relates to the classification and/or monitoring of individuals suffering from a cardiovascular disease or disorder, such as especially hearth disease caused by atherosclerosis, according to survival prognoses based on the determination of the levels of biomarker YKL-40 molecules or fragments hereof in sample taken from said individuals. The found YKL-40 levels are compared to reference levels in order to classify and/or monitor the state of the individual. Furthermore the level of YKL-40 may be used to predict risk of disease relapse.

YKL-40 is a new prognostic biomarker of survival. YKL-40 levels may be measured in any type of biological sample such as a serum, blood or plasma sample and the YKL-40 measured may be protein, fragments or peptides hereof or any other transcriptional product of the YKL-40 encoding gene.

It is an object of the present invention to provide a method for classifying individuals suffering from cardiovascular disease, such as especially hearth disease caused by atherosclerosis, according to a prognosis of their survival, said method comprising: measuring the level of YKL-40 in a biological sample from said individual, and comparing the measured level to a reference level of YKL-40.

It is further an object of the present invention to provide a method for monitoring the health state of an individual suffering from cardiovascular disease, such as a hearth disease caused by atheroslerosis, in relation to a prognosis of their survival, said method comprising: measuring the level of YKL-40 in a biological sample from said individual; and comparing the measured level to a reference level of YKL-40. The reference level of YKL-40 may be any reference level as described herein, and especially as described in the section “reference levels”. Further details for this method of the present invention will be apparent from the text describing the above mentioned first method relating to “classifying individuals”. Accordingly, any features mentioned in relation to the first method of the invention apply mutatis mutandis to this further method of the invention, unless otherwise stated.

It is an additional object of the present invention to provide a kit of parts comprising a method of detecting YKL-40 in a biological sample and instruction on how to classify and/or monitor individuals according to their YKL-40 levels.

The present invention furthermore provides an embodiment consisting of a kit of parts comprising a method of detecting YKL-40 in a biological sample and instruction on how to classify and/or monitor individuals according to their YKL-40 levels as well as methods for detecting additional biomarkers.

### BRIEF DESCRIPTION OF DRAWINGS

FIG. 1 Demographic data of individuals in the YKL-40 study.

FIG. 2 Hazard ratios.

FIG. 3 Hazard ratios including intervention indicator and risk factors.

FIG. 4 Survival curve according cut-off value classification.

FIG. 5 Survival curve according to classification.

FIGS. 6A and B Dipstick embodiments seen from above.

FIG. 7 Effect of f(YKL-40) on time to death, to cardiovascular death and myocardial infarction alone or in combination with risk factors plus selected indicators of treatment.

### DETAILED DESCRIPTION OF THE INVENTION

Definitions

The following definitions are provided to simplify discussion of the invention. They should not, therefore, be construed as limiting the invention, which is defined in scope by the appended claims and the following description.

Ameliorate: To improve or make better; in association with a disease state a lessening in the severity or progression of a disease state, including remission or cure thereof, alternatively the perceived lessening of severity such as lessening of associated pain.

Antibody: Immunoglobulin molecules and active portions or fragments of immunoglobulin molecules such as Fab and F(ab′).sub.2 which are capable of binding an epitopic determinant of the YKL-40 protein. Antibodies are for example intact immunoglobulin molecules or fragments thereof retaining the immunologic activity.

Antigen: An immunogenic full-length or fragment of a YKL-40 molecule.

Biological sample: A sample obtained from an individual.

Biomarker: A molecular indicator of a specific biological property, such as a particular pathological or physiological state. The term “marker” is used synonymously herewith herein.

Cardiovascular disease: The term cardiovascular disease refers to the class of diseases that involve the heart or blood vessels (arteries and veins). The term refers to any disease that affects the cardiovascular system and the consequences of cardiovascular disease and is used as such herein. There are many types of cardiovascular diseases including, but not limited to: acute coronary syndrome, acute myocardial infarction (AMI/STEMI/ST-elevation), myocardial infarction (heart attack), unstable angina pectoris/UAP/non-ST-elevation myocardial infarction, aneurysms, angina, atherosclerosis, coronary artery disease (CAD), ischemic heart disease, ischemic myocardium, arrhythmia, atrial fibrillation, cardiac arrhythmia, ventricular tachycardia, ventricular fibrillation, cardiac and sudden cardiac death, cardiomyopathy, congestive heart failure, heart failure, diastolic and systolic ventricular dysfunction, dilated cardiomyopathy, high blood pressure (hypertension), hypertrophic cardiomyopathy, valve disease, mitral valve prolapse, mitral valve regurgitation and/or stenosis, aortic valve regurgitation and/or stenosis, myocarditis and venous thromboembolism.

Disease state: An illness or injury in an individual.

Disorder: An illness or injury in an individual often of a congenital type.

hnRNA: heteronuclear RNA

Individual: a single member of a species, herein preferably a mammalian species.

mAb: monoclonal antibody

Mammal: as used herein includes both humans and non-humans.

mRNA: messenger RNA

Patient: Any individual suffering from a disease or disorder.

RNA: Any type of RNA originating alternatively isolated from nature or synthesized.

Stable coronary artery disease: The term “stable coronary artery disease” refers to coronary artery diseases or atherosclerotic hearth diseases caused by the accumulation of atheromatous plaques within the walls of the coronary arteries that supply the myocardium (the muscle of the heart) with oxygen and nutrients, wherein the diseases gives cause to stable symptoms or signs of said diseases.

Substantially pure: as used to describe YKL-40, refers to the substantially intact molecule which is essentially free of other molecules with which YKL-40 may be found in nature.

Classification of Individuals

Cardiovascular diseases are the leading cause of death globally. Atherosclerosis is the major cause of cardiovascular diseases and as the life style that facilitates the occurrence of atherosclerosis continues to spread across the continents, cardiovascular diseases are predicted to remain the leading cause of death in the future. Providing the best possible treatment for individuals suffering from cardiovascular disease is therefore of interest both to individual suffering here from but also to the medical institutions that are to treat an ever growing number of these patients.

The best possible treatment is a treatment tailored to each individual. For example, it has been known that persons suffering from coronary artery disease (CAD), a disease associated with a high death rate, respond differently to the same treatment, but there has been no method to monitor the effect of a given treatment or to differentiate these patients prior to a fatal occurrence. The present invention resolves this problem, as it both provides a classification system that allows each individual to be classified according to a prognosis of survival and provides a method of monitoring the individuals over time. The classification and monitoration is based on the measurement of YKL-40 levels in biological samples taken from the individuals to be classified/monitored and comparing the found levels with that of a reference level. This enables a prognosis for the survival of the individual and thus is of assistance in determining the intensity of the treatment the individual should receive and whether the administered treatment is sufficient or inadequate.

Tailoring the treatment to each individual by the classification according to survival prognosis will improve both the ameliorative and the curative effect of the administered treatment, improve the survival rate of the patients as whole, lower relapse risks, and heighten the quality of life following the occurrence of a cardiovascular disease. Furthermore, there will be a financial benefit in that the amount of drugs administered may be adjusted acutely. Also, the ability to monitor this group of individuals will be of assistance in choosing the most effective immediate and follow-up treatment, and be of guidance when counseling on lifestyle chances required subsequent to the occurrence of a cardiovascular disease or disorder.

A statistically increased level of YKL-40 is indicative of an increased risk of death as can be seen in the Examples. YKL-40 is thus a biomarker that allows a prognosis of survival for the individual for which the YKL-40 level has been determined. The prognosis may be correlated with other signs of health status known to those skilled in the clinical arts. If the level of YKL-40 is increased to a statistically significant level a prognosis of death or reduced survival may be issued.

Where it is of interest to monitor a individual for example in order to assess the effectiveness of a treatment such as the amelioration of a disease YKL-40 levels in a biological assay sample taken from the individual (blood, serum or other) should be measured before (for background) and periodically during the course of treatment. Because reductions or increases in YKL-40 levels may be transient, the assay will preferably be performed at regular intervals (e.g., every week) as well as prior to and after each treatment. Depending on the course of treatment, severity of the case and other clinical variables, clinicians of ordinary skill in the art will be able to determine an appropriate schedule for assaying YKL-40 levels for the purpose of monitoring the disease and/or treatment of a particular individual.

Reference Levels

A statistically increased level of YKL-40 is indicative of survival and may as described herein be used in the classification and or monitoration of individuals suffering from heart disease. Whether the YKL-40 level of a given individual is increased or not may be asserted by correlation of the measured value with that of a reference level. The group of individuals who form the basis for the calculation of the reference level may be a group of healthy individuals of various ages or may be an age specific group. Healthy individuals are individuals who at the time of sampling are not diagnosed with heart related diseases or disorders.

An age specific group of individuals may comprise individuals that are all born within the same year or decade or any other groupings such as groups comprising individuals that are of 0 to 10 years of age, 10 to 20 years of age, 20 to 30 years of age, 30 to 40 years of age, 40 to 50 years of age, 50 to 60 years of age, 60 to 70 years of age, 70 to 80 years of age, 80 to 90 years of age, 90 to 100 years of age, and so on. The intervals may span 2 years of age difference, 3, 4, or 5 years of age difference, 6, 7, 8, 9, 10 years of age difference (as written), 12 15, 20 or more years of age difference. The intervals may furthermore be open ended e.g. the individuals are all above the age of 20, 30, 40, 50, 60 or other.

An age varied or age-specific group of individuals sampled for obtaining a YKL-40 reference level may furthermore be individuals suffering from a disease such as a heart disease or disorder and who either are displaying symptoms hereof or not, or having previously suffered from such a disease or disorder and are considered cured hereof. The heart disease or disorder may for example be coronary artery disease or any of the abovementioned heart diseases or disorders. The group of individuals who form the basis for the calculation of the reference level may furthermore be a group of individuals of mixed sex or same sex. Reference levels may also be obtained from the same individual as is presently suffering from a heart disease or disorder for example may YKL-40 levels be measured in one or more samples obtained prior to diagnosis of the disease or disorder (pre-illness) and or prior to the establishment of symptoms of the disease or disorder (pre-symptom).

The classification of individuals based on their YKL-40 levels may be performed according to the results described in the Examples. As can be seen from these there is a relationship between increased YKL-40 levels and increased hazard ratio. Hazard ratios indicate increased risk of death and are calculated as known to those skilled in the art. In the present examples the hazard ratio in the survival analysis is the effect of an explanatory variable unstable angina, myocardial infarction, cardiac death or total death on the hazard or risk of an event. Accordingly, the hazard ratio of a certain value of YKL-40 indicates increased risk of e.g. myocardial infarction (MI), cardiovascular death or all cause death.

One method of classification is the use of a cut-off value as a reference value. A cut-off value is a value the typically divides a number of individuals into two groups: those that have an YKL-40 level above a specific cut-off value, and those that have an YKL-40 level below the specified cut-off value. The cut-off value may be any value that represents a physiological YKL-40 level as measured in any type of biological sample, either as chosen by a person skilled in the art.

The cut-off value may be used as a yes or no indicator of whether an individual is at increased risk. The increased risk may be an increased risk of disease such as heart disease; specifically heart disease caused by atherosclerosis, specifically a heart disease the individual previously or currently is suffering from, alternatively the risk may be an increased risk of a short survival such as given by an increased YKL-40 level/an YKL-40 level above the cut-off value.

In one embodiment of the methods according to the invention, the reference level of YKL-40 is a cut-of value of about 80 μg/l, such as e.g. specifically 82 μg/l. As can be seen from the examples herein the present inventors have surprisingly found that YKL-40 values below about 80 μg/l does not correlate with the hazard ratios, whereas YKL-40 levels above said value correlates with the hazard ratios for cardiovascular death, myocardial infarction (MI) and all cause mortality. Accordingly, the present inventors have found that YKL-40 levels above about 80 μg/l can be used for classifying individuals suffering from hearth disease caused by atherosclerosis, such as e.g. stable coronary artery disease, according to a prognosis of their survival by measuring the level of YKL-40 in a biological sample from said individual, and comparing the measured level to a reference level of YKL-40.

The prognosis of survival as mentioned in the methods according to the present invention may specifically be a prognosis of suffering cardiovascular death, and more preferably a prognosis of the risk of suffering a myocardial infarction.

A cut-off value may therefore be any value selected from the following group of values, or fall in between any of the mentioned values: 80 μg/l serum YKL-40, 90 μg/l, 95 μg/l, 100 μg/l, 105 μg/l, 110 μg/l, 115 μg/l, 120 μg/l, 125 μg/l, 130 μg/l, 140 μg/l, 150 μg/l, 160 μg/l, 170 μg/l, 180 μg/l, 190 μg/l, and/or 200 μg/l, serum YKL-40. In one embodiment of the methods according to the invention the cut-off value is any of the following values: 100 μg/l serum YKL-40, 105 μg/l, 106 μg/l, 107 μg/l, 108 μg/l, 109 μg/l, 110 μg/l, 110 μg/l, 111 μg/l, 112 μg/l, 113 μg/l, 114 μg/l, 115 μg/l, 120 μg/l serum YKL-40. In a specific embodiment the cut-off value is 110 μg/l serum YKL-40.

In addition to the above-mentioned cut-off value of about 80 μg/l, individuals may be further classified in groups according to their YKL-40 level, this may for instance be performed by the following set of cut-off values, where an increased YKL-40 value, i.e. a cut-off value, indicates a more severe/advanced stage of the hearth disease in question: about 80 μg/l, about 90 μg/l, about 100 μg/l, about 110 μg/l, about 120 μg/l, about 130 μg/l, about 140 μg/l, about 150 μg/l, about 160 μg/l, about 170 μg/l, about 180 μg/l, about 190 μg/l, about 200 μg/l, about 210 μg/l, and about 220 μg/l.

Alternatively, based hereon, as an example, individuals may be grouped according to their YKL-40 levels in increments of 20 so that: group 0 individuals have serum YKL-40 levels of less than 90 μg/l (microgram/liter), group I individuals have serum YKL-40 levels of 100 μg/l+/−10 μg/l, group 2 individuals have serum YKL-40 levels of 120 μg/l+/−10 μg/l, group 3 individuals have serum YKL-40 levels of 140 μg/l+/−10 μg/l, group 4 individuals have serum YKL-40 levels of 160 μg/l+/−10 μg/l, group 5 individuals have serum YKL-40 levels of 180 μg/l+/−10 μg/l, group 6 individuals have serum YKL-40 levels of 200 μg/l+/−10 μg/l, group 7 individuals have serum YKL-40 levels of 220 μg/l+/−10 μg/l, group 8 individuals have serum YKL-40 levels of 240 μg/l+/−10 μg/l, group 9 individuals have serum YKL-40 levels of 260 μg/l+/−10 μg/l, group 10 individuals have serum YKL-40 levels of 280 μg/l+/−10 μg/l, group 11 individuals have serum YKL-40 levels of 300 μg/l+/−10 μg/l, group 12 individuals have serum YKL-40 levels of 320 μg/l+/−10 μg/l, group 13 individuals have serum YKL-40 levels of 340 μg/l+/−10 μg/l, group 14 individuals have serum YKL-40 levels of above 350 μg/l. In the given example serum YKL-40 levels have been used, however, YKL-40 levels obtained from other biological samples and measured as protein, RNA or other as herein mentioned also fall within the scope of the present invention. Furthermore, the increments between the groups may be of 2 μg/l, such as 4, 5, 6, 8, 10, 15, 20, 25, 30, 35, 40, 45, 50, 55, 60, 75, 80, 95, 90 or 100 μg/l YKL-40. Preferably, the increments are 20 or 30 μg/l YKL-40 as measured in serum. The increments of 20 or 30 μg/l YKL-40 may start at 50 μg/l serum YKL-40.

Alternatively to the example given above, the classification of individuals may be done in groups that commence at a lower serum YKL-40 level than indicated above such as a group 0 comprising individuals with serum YKL-40 levels of 40 μg/l+/−5 μg/l, and group 1 individuals have serum YKL-40 levels 50 μg/l+/−5 μg/l, group 2 individuals have serum YKL-40 levels of 60 μg/l+/−5 μg/l, group 3 individuals have serum YKL-40 levels of 70 μg/l+/−5 μg/l, group 4 individuals have serum YKL-40 levels of 80 μg/l+/−5 μg/l, group 5 individuals have serum YKL-40 levels of 90 μg/l+/−5 μg/l, and group 6 individuals have serum YKL-40 levels of 100 μg/l+/−5 μg/l and so on. The preferred groupings for the purpose of classification may be related to the age of the individuals to be classified as well disease state, future treatments and other.

A further example of a classification scheme is shown in the table below. In this embodiment the groups are characterized by a concentration range of YKL-40 as measured in a biological sample. The ranges given in the example span increments of 25 μg/l, but may span smaller increments such as 5, 10, 15 or 20 μg/l, or alternatively span larger increments such as 30, 35, 40, 45 or 50, 60, 70 80 90 or 100 μg/l.

| Group | Serum YKL-40 μg/l |
|---|---|
| 1 | <85 |
| 2 | 85-110 |
| 3 | 110-135 |
| 4 | 135-160 |
| 5 | 160-185 |
| 6 | 185-210 |
| 7 | 210-235 |
| 8 | 235-260 |
| 9 | 260-285 |
| 10 | >285 |

For all the above and below mentioned classification groupings, it applies that the higher the YKL-40 level, the more severe/advanced is the cardiovascular disease, and the worse is the survival prognosis.

Normal YKL-40 values for healthy individuals may preferably be used as reference levels for comparing a measured level of YKL-40. When such normal values are used it is furthermore possible to include an age adjustment, or e.g. a classification according to severity. Accordingly, age adjusted reference levels obtained from healthy individuals and as preferably described in co-pending application with the title “YKL-40 as a general marker for non-specific disease” and especially in the section termed “reference levels” may be used as the reference levels in the methods according to the present invention.

Due to the relationship between YKL-40 levels in serum and the associated hazard rations, the individuals to be classified may also be classified according to the calculated hazard ratios. A group of individuals may also be classified according to percentiles, such that the total group 100% and the 10% of the group with the lowest YKL-40 levels are group 1, the second lowest 10% percentile is group 2 and so forth. The percentiles may be 1%, 2%, 3%, 4%, 5%, 6%, 7%, 8%, 9%, 10%, 11%, 12%, 12.5%, 13%, 14%, 15%, 20%, 25%, 30%, 33% or 35% percentile groupings, or any percentile falling between or above the mentioned percentiles. An example of 10% percentile groupings is given in the Examples.

The present invention provides a method for classifying individuals suffering from heart disease caused by atherosclerosis according to a prognosis of their survival, said method comprising: measuring the level of YKL-40 in a biological sample from said individual, and comparing the measured level to a cut-off value. The cut-off value being a level of YKL-40, for example, a level of YKL-40 in a biological sample, such a level of serum YKL-40. The YKL-40 levels may be any of the one or more levels of YKL-40 described herein. The prognosis of survival may specifically be a prognosis of suffering a cardiovascular death, and more preferably a prognosis of the risk of suffering a myocardial infarction.

A specific embodiment of the present invention relates to a method for classifying individuals suffering from atherosclerotic coronary artery disease according to a prognosis of their risk of suffering a myocardial infarction, said method comprising: measuring the level of YKL-40 in a biological sample from said individual, and comparing the measured level to one or more cut-off values; preferably one of the one or more cut-off values is a value of about 80 μg/l.

The present invention further provides a method for classifying individuals according to a prognosis of their survival, said method comprising: measuring the level of YKL-40 in a biological sample from said individual; and comparing the measured level to a reference level of YKL-40. A statistically significant increase is an indicator for shorter survival of the individual. The individual may be suffering from any type of disease such as a cardiovascular disease. Specifically the individual may be suffering from coronary artery disease.

Monitoring of Individuals

The present invention relates to the monitoring of individuals based on the prognosis of their survival as measured from their YKL-40 levels. Monitoring individuals according to the measured YKL-40 levels may be used as an indication of the general state of health of an individual and/or as an indication of the effectiveness of an administered treatment. The individuals or patients may be suffering from a disease or disorder such as a cardiovascular disease or disorder. Specifically the individual or patient may be suffering from coronary artery disease.

Monitoring YKL-40 levels as a prognosis of death in individuals suffering from cardiovascular disorders and diseases facilitates administration of the most optimal treatment for each individual. The administration of an effective treatment improves both the ameliorative and curative effect of the administered treatment as well as the survival chances of the individuals, and lessens relapse risks. Thus, YKL-40 can be used for monitoring the sufficiency of medical treatment of patients with stable coronary artery disease, and hereby assist in the reduction of the high occurrence of non-fatal and fatal cardiovascular events in these patients. Furthermore, the administration of the most effective treatment is also an issue when assessing the cost/benefits of the given treatment.

Therefore it is an aspect of the present invention to provide a method for monitoring the health state of an individual in relation to a prognosis of their survival, said method comprising: measuring the level of YKL-40 in a biological sample from said individual; and comparing the measured level to a reference level of YKL-40; wherein a statistically significant increase is an indicator for shorter survival of the individual.

The present invention furthermore relates to a method of treating an individual suffering from a coronary artery disease, comprising measuring the level of YKL-40 in a biological sample from said individual, and based on the measured level selecting a medicament, and administering a sufficient amount of said medicament to said individual. The coronary artery disease is preferably stable coronary artery disease, and the comparison is preferably performed by comparing with one or more reference levels as described herein above.

Heart Diseases and Disorders

The term cardiovascular disease refers to the class of diseases that involve the heart or blood vessels (arteries and veins). The term refers to any disease that affects the cardiovascular system and the consequences of cardiovascular disease and is used as such herein, although it generally is used in relation to atherosclerosis. There are many types of cardiovascular diseases including: acute coronary syndrome, acute myocardial infarction (AMI/STEMI/ST-elevation), myocardial infarction (heart attack), unstable angina pectoris/UAP/non-ST-elevation myocardial infarction, aneurysms, angina, atherosclerosis, coronary artery disease (CAD), ischemic heart disease, ischemic myocardium, arrhythmia, atrial fibrillation, cardiac arrhythmia, ventricular tachycardia, ventricular fibrillation, cardiac and sudden cardiac death, cardiomyopathy, congestive heart failure, heart failure, diastolic and systolic ventricular dysfunction, dilated cardiomyopathy, high blood pressure (hypertension), hypertrophic cardiomyopathy, valve disease, mitral valve prolapse, mitral valve regurgitation and/or stenosis, aortic valve regurgitation and/or stenosis, myocarditis and venous thromboembolism, all of which are of relevance for the present invention.

Of special interest to the present invention are the following diseases which in the below are described in more detail: atherosclerotic coronary artery disease (CAD), atherosclerotic coronary heart disease, atherosclerotic cardiovascular disease, ischemic myocardial disease, ischemic coronary artery disease, ischemic heart failure, ischemic heart disease, ischemic cardiac arrhythmia, non-fatal acute myocardial infarction, sudden coronary death (cardiac death), and fatal and non-fatal acute myocardial infarction, acute coronary syndrome, acute myocardial infarction/AMI/STEMI/ST-elevation, atrial fibrillation, cardiac arrhythmia, cardiomyopathy, congestive heart failure, ischemic myocardium, myocardial infarction, unstable angina pectoris/UAP/non-ST-elevation, myocardial infarction, ventricular tachycardia and ventricular fibrillation. Of most particular interest to the present invention is any heart/cardiovascular diseases or disorders associated with atherosclerosis.

Stable angina can be described as chest pain or discomfort that typically occurs with activity or stress, where the episodes of pain or discomfort are provoked by similar or consistent amounts of activity or stress. Unstable angina pectoris (UAP) can be described as angina pectoris that occurs unpredictably or suddenly increases in severity or frequency; attacks may occur without provocation, such as during sleep or rest, and which may not respond to nitroglycerin, and may be of unusually long duration. Unstable angina pectoris is also considered an initial stage up to acute myocardial infarction, which again may lead to cardiovascular death. UAP is however often difficult to register due to the lack of actual physiologically measurable parameters. Acute myocardial infarction may furthermore be grouped according to the appearance of the electrocardiogram (ECG/EKG), i.e. as non-ST segment elevation myocardial infarction (NSTEMI) or as ST segment elevation myocardial infarction (STEMI).

One specific embodiment of the invention relates to the method for classifying individuals suffering from heart disease caused by atherosclerosis, according to their survival, wherein the hearth disease is not unstable angina pectoris, said method comprising: measuring the level of YKL-40 in a biological sample from said individual, and comparing the measured level to a reference level of YKL-40. The reference level is any reference level as described herein, and especially as described in the section “reference levels”.

Another specific embodiment of the invention relates to the method for monitoring the health state of an individuals suffering from heart disease caused by atherosclerosis, according to their survival, wherein the hearth disease is not unstable angina pectoris, said method comprising: measuring the level of YKL-40 in a biological sample from said individual, and comparing the measured level to a reference level of YKL-40. The reference level is any reference level as described herein, and especially as described in the section “reference levels”.

Atherosclerosis

Atherosclerosis is a disease affecting arterial blood vessels. It is a chronic inflammatory response in the walls of arteries, in large part due to the deposition of lipoproteins. It is caused by the formation of multiple plaques within the arteries. Atherosclerosis causes two main problems. First, the atheromatous plaques, though long compensated for by artery enlargement, eventually lead to plaque ruptures and stenosis of the artery and, therefore, an insufficient blood supply to the organ it feeds. If the compensating artery enlargement process is excessive, a net aneurysm results. These complications are chronic, slowly progressing and cumulative. Most commonly, soft plaque suddenly ruptures, causing the formation of a thrombus that will rapidly slow or stop blood flow, that is, within 5 minutes, leading to death of the tissues fed by the artery. This catastrophic event is an infarction. One of the most common recognized scenarios is coronary thrombosis of a coronary artery, causing myocardial infarction (a heart attack). Another common scenario in very advanced disease is claudication from insufficient blood supply to the legs, typically due to a combination of both stenosis and aneurysmal segments narrowed with clots. Since atherosclerosis is a body-wide process, similar events occur also in the arteries to the brain, intestines, kidneys, legs, etc.

Inflammation plays an important role in atherogenesis and atherothrombotic events and is associated with the development of myocardial infarction, stroke and cardiovascular mortality. Furthermore, all vascularized tissues exposed to injury display a reaction of repair involving altered collagen turnover and inflammation. As indicated by the finding that YKL-40 is produced by macrophages and neutrophils locally in tissues with inflammation, YKL-40 is a new biomarker of acute and chronic inflammation in individuals with cardiovascular diseases and disorders such as coronary artery disease.

Coronary Artery Disease

Coronary artery disease (CAD) is the most common form of heart disease in the Western world. Coronary artery disease, also called atherosclerotic coronary artery disease, coronary heart disease (HAD), atherosclerotic coronary heart disease, atherosclerotic cardiovascular disease, ischemic heart disease, and atherosclerotic heart disease, is the end result of the accumulation of atheromatous plaques within the walls of the arteries that supply the myocardium with oxygen and nutrients. While the symptoms and signs of coronary heart disease are noted in the advanced state of disease, most individuals with coronary heart disease show no evidence of disease for decades as the disease progresses before the first onset of symptoms, often a “sudden” heart attack, finally arise. After decades of progression, some of these atheromatous plaques may rupture and (along with the activation of the blood clotting system) start limiting blood flow to the heart muscle.

Coronary artery disease may manifest itself at different extents. It may affect a single coronary artery, or vessel, supplying the myocardium, or it may affect two vessels, three vessels or more. The severity of the disease increases with an increasing number of affected vessels. Furthermore the severity of the disease depends on whether the affected vessel is an end vessel or a collateral vessel. A collateral vessel is a vessel supplying a region of a tissue such as a region of the myocardium together with another vessel, there thus being two or more vessels supplying the same tissue region. An end vessel is a vessel that singularly supplies a specific tissue region, such as a specific region of the myocardium. Thus, the disease is more severe if an end vessel is affected than if a collateral vessel is affected.

In a preferred embodiment of the methods according to the invention the individual suffers from atherosclerotic coronary artery disease, in a more preferred embodiment of the present invention the hearth disease is stable coronary artery disease.

Acute Coronary Syndrome

An acute coronary syndrome (ACS) is a set of signs and symptoms, usually a combination of chest pain and other features, interpreted as being the result of abruptly decreased blood flow to the heart (cardiac ischemia); the most common cause for this is the disruption of atherosclerotic plaque in an epicardial coronary artery. Acute coronary syndrome often reflects a degree of damage to the coronaries by atherosclerosis. The subtypes of acute coronary syndrome include unstable angina (UA, not associated with heart muscle damage), and two forms of myocardial infarction (heart attack), in which heart muscle is damaged. These types are named according to the appearance of the electrocardiogram (ECG/EKG) as non-ST segment elevation myocardial infarction (NSTEMI) and ST segment elevation myocardial infarction (STEMI).

Acute myocardial infarction (AMI or MI), more commonly known as a heart attack, is a medical condition that occurs when the blood supply to a part of the heart is interrupted, most commonly due to rupture of a vulnerable plaque. The outcome of an acute myocardial infarction may be fatal or non-fatal to the individual. The resulting ischemia or oxygen shortage causes damage and potential death of heart tissue. Important risk factors are a previous history of vascular disease such as atherosclerotic coronary heart disease and/or angina, a previous heart attack or stroke, any previous episodes of abnormal heart rhythms or syncope, older age—especially men over 40 and women over 50, smoking, excessive alcohol consumption, the abuse of certain drugs, high triglyceride levels, high LDL (“Low-density lipoprotein”) and low HDL (“High density lipoprotein”), diabetes, high blood pressure, obesity, and chronically high levels of stress in certain persons.

ACS should be distinguished from stable angina, which develops during exertion and resolves at rest. In contrast with stable angina, unstable angina occurs suddenly, often at rest or with minimal exertion, or at lesser degrees of exertion than the individual's previous angina (“crescendo angina”). New onset angina is also considered unstable angina, since it suggests a new problem in a coronary artery.

It is an object of the present invention to provide a method of classifying and/or monitoring one or more individuals suffering from unstable angina or myocardial infarction based on the prognosis of their survival measured as increased YKL-40 levels in a sample obtained from said individual(s). More preferably classifying and/or monitoring one or more individuals suffering from myocardial infarction (MI). The unstable angina is considered an initial stage of MI.

Ischemic Cardiomyopathy

Ischemic cardiomyopathy (also known as ischemic heart disease (IHD)) and related to ischemic myocardial disease, ischemic coronary artery disease, ischemic heart failure, ischemic heart disease, ischemic cardiac arrhythmia, and ischemic myocardium) is a weakness in the muscle of the heart due to inadequate oxygen delivery to the myocardium with coronary artery disease (atherosclerosis of the coronary arteries) being the most common cause. Anemia and sleep apnea are relatively common conditions that can contribute to ischemic myocardium and hyperthyroidism can cause a ‘relative’ ischemia secondary to high output heart failure. Individuals with ischemic cardiomyopathy typically have a history of myocardial infarction (heart attack), although longstanding ischemia can cause enough damage to the myocardium to precipitate a clinically significant cardiomyopathy even in the absence of myocardial infarction. In a typical presentation, the area of the heart affected by a myocardial infarction will initially become necrotic as it dies, and will then be replaced by scar tissue (fibrosis). This fibrotic tissue is akinetic; it is no longer muscle and cannot contribute to the heart's function as a pump. If the akinetic region of the heart is substantial enough, the affected side of the heart (i.e. the left or right side) will go into failure, and this failure is the functional result of an ischemic cardiomyopathy. Symptoms of stable ischemic heart disease include angina and decreased exercise tolerance. Unstable IHD presents itself as chest pain or other symptoms at rest, or rapidly worsening angina. Diagnosis of IHD is with an electrocardiogram, blood tests (cardiac markers), cardiac stress testing or a coronary angiogram. Depending on the symptoms and risk, treatment may be with medication, percutaneous coronary intervention (angioplasty) or coronary artery bypass surgery (CABG). Many diseases can result in cardiomyopathy. These include diseases like hemochromatosis, amyloidosis, diabetes, hyperthyroidism, lysosomal storage diseases and the muscular dystrophies.

It is an object of the present invention to provide a method for classifying and or monitoring one or more individuals suffering from any of the above mentioned ischemic cardiomyopathies according to a prognosis of their survival, said method comprising: measuring the level of YKL-40 in a biological sample from said individual(s), and comparing the measured level to a reference level of YKL-40. The reference level may be any of the YKL-40 levels as described herein, especially as described in the section “reference levels”. The levels of YKL-40 may be measured in any of the types of assays herein disclosed such as an immunoassay or a PCR based assay and in any type of biological sample, especially serum, plasma or blood samples.

Heart Failure

Congestive heart failure (CHF), congestive cardiac failure (CCF) or just heart failure is a condition that can result from any structural or functional cardiac disorder that impairs the ability of the heart to fill with or pump a sufficient amount of blood through the body. It is not to be confused with “cessation of heartbeat”, which is known as asystole, or with cardiac arrest, which is the cessation of normal cardiac function with subsequent hemodynamic collapse leading to death. Because not all individuals have volume overload at the time of initial or subsequent evaluation, the term “heart failure” is preferred over the older term “congestive heart failure”. Heart failure is often undiagnosed due to a lack of a universally agreed definition and difficulties in diagnosis, particularly when the condition is considered “mild”.

It is an object of the present invention to provide a method for classifying and or monitoring one or more individuals suffering from heart failure according to a prognosis of their survival, said method comprising: measuring the level of YKL-40 in a biological sample from said individual(s), and comparing the measured level to a reference level of YKL-40.

Cardiac Arrest

A cardiac arrest, also known as cardio respiratory arrest, cardiopulmonary arrest or circulatory arrest, is the abrupt cessation of normal circulation of the blood due to failure of the heart to contract effectively during systole. “Arrested” blood circulation prevents delivery of oxygen to all parts of the body. Cerebral hypoxia, or lack of oxygen supply to the brain, causes victims to lose consciousness and to stop normal breathing, although agonal breathing may still occur. Brain injury is likely if cardiac arrest is untreated for more than 5 minutes, although new treatments such as induced hypothermia have begun to extend this time. To improve survival and neurological recovery immediate response is paramount. Cardiac arrest is a medical emergency that, in certain groups of individuals, is potentially reversible if treated early enough. When unexpected cardiac arrest leads to death this is called sudden cardiac death (SCD) alternatively named sudden coronary death. The primary first-aid treatment for cardiac arrest is cardiopulmonary resuscitation (commonly known as CPR) to provide circulatory support until availability of definitive medical treatment, which will vary dependant on the rhythm the heart is exhibiting, but often requires defibrillation.

The most frequent underlying cause of cardiac arrest and sudden cardiac death is coronary artery disease, other categories of causes include: non-atherosclerotic coronary artery abnormalities, hypertrophy of ventricular myocardium, myocardial diseases and heart failure, including arrhythmogenic right ventricular cardiomyopathy, hypertrophic cardiomyopathy, dilated cardiomyopathy, myocardial infarction, non-compaction cardiomyopathy, inflammatory, infiltrative, neoplastic, and degenerative processes, diseases of the cardiac valves, congenital heart disease, primary electrophysiological abnormalities, such as Long QT syndrome, both congenital and acquired, sick sinus syndrome, Brugada syndrome, catecholaminergic polymorphic ventricular tachycardia, rhythm instability related to neurohumoral and central nervous system influences, sudden infant death syndrome and sudden death in children, commotio cordis, mechanical interference with venous return, aortic dissection, and toxic/metabolic disturbances.

It is an object of the present invention to provide a method for monitoring and/or classifying individuals suffering from congestive heart failure according to a prognosis of their survival, said method comprising: measuring the level of YKL-40 in a biological sample from said individual, and comparing the measured level to a reference level of YKL-40. The biological sample may be a blood, serum or plasma sample.

Cardiac Arrhythmia

Cardiac arrhythmia is any of a group of conditions in which the electrical activity of the heart is irregular or is faster or slower than normal. Some arrhythmias are life-threatening medical emergencies that can cause cardiac arrest and sudden death. Others cause aggravating symptoms, such as an awareness of a different heart beat, or palpitation, which can be annoying. Some are quite minor and can be regarded as normal. A list of common cardiac arrhythmias of interest to the present invention include, but are not limited to: atrial rhythms, premature atrial contractions (PACs), wandering atrial pacemaker, multifocal atrial tachycardia, supraventricular tachycardia (SVT), atrial flutter, atrial fibrillation (Afib), ventricular rhythms, premature ventricular contractions (PVC), accelerated idioventricular rhythm, ventricular tachycardia (VT), ventricular fibrillation (VF), polymorphic ventricular tachycardia, ventricular extra beats, atrial ventricular arrhythmias, AV nodal reentrant tachycardia, AV reentrant tachycardia, Wolff-Parkinson-White syndrome, Lown-Ganong-Levine syndrome, junctional arrhythmias, junctional rhythm, junctional tachycardia, premature junctional complex, heart blocks, also known as AV blocks, first degree heart block, also known as PR prolongation, second degree heart block, Type 1 second degree heart block, also known as Mobitz I or Wenckebach, Type 2 second degree heart block, also known as Mobitz II, third degree heart block, also known as complete heart block, and less common arrhythmias such as Trigeminal rhythm.

Of special interest to the present invention are the following arrhythmias: ventricular tachycardia, ventricular fibrillation and atrial fibrillation, each of which is further described in the below.

Ventricular tachycardia (V-tach or VT) is a tachycardia, or fast heart rhythm that originates in one of the ventricles of the heart. This is a potentially life-threatening arrhythmia because it may lead to ventricular fibrillation and sudden death. Ventricular tachycardia can be classified based on its morphology, duration of the episodes or on the basis of the symptoms. Some VT is associated with reasonable cardiac output and may even be asymptomatic. The heart usually tolerates this rhythm poorly in the medium to long term, and individuals may certainly deteriorate to pulseless VT or to VF.

Ventricular fibrillation (V-fiB or VF) is a condition in which there is uncoordinated contraction of the cardiac muscle of the ventricles in the heart. As a result, the heart fails to adequately pump blood; hypoxia soon occurs, followed by unconsciousness within twenty to thirty seconds. Ventricular fibrillation is a medical emergency. If the arrhythmia continues for more than a few seconds, blood circulation will cease—as evidenced by lack of pulse, blood pressure, and respiration—and eventually death will occur. Ventricular fibrillation is a cause of cardiac arrest and sudden cardiac death.

Atrial fibrillation (AF or afib) is a cardiac arrhythmia that involves the two upper chambers (atria) of the heart. It is defined as being irregularly irregular, and can often be identified as such when taking a pulse. Atrial fibrillation is the most common arrhythmia; risk increases with age, with 8% of people over 80 having AF. In atrial fibrillation, the electrical impulses that are normally generated by the sinoatrial node are replaced by disorganized activity in the atria, leading to irregular conduction of impulses to the ventricles that generate the heartbeat. The result is an irregular heartbeat. This may be continuous (persistent or permanent AF) or alternating between periods of a normal heart rhythm (paroxysmal AF). The natural tendency of atrial fibrillation is to become a chronic condition. Chronic AF leads to an increased risk of death. Atrial fibrillation is often asymptomatic, and is not in itself generally life-threatening, but may result in palpitations, fainting, chest pain, or congestive heart failure. Individuals with atrial fibrillation are at significantly increased chance of stroke (about 2 to 7 times the regular population), and AF is a leading cause of stroke.

It is an object of the present invention to provide a method for monitoring and/or classifying individuals suffering from cardiac arrhythmias according to a prognosis of their survival, said method comprising: measuring the level of YKL-40 in a biological sample from said individual, and comparing the measured level to a reference level of YKL-40. The reference level may be any of the YKL-40 levels as described herein, especially as described in the section “reference levels”. Of particular interest to the present invention are cardiac arrhythmias such as ventricular tachycardia, ventricular fibrillation and atrial fibrillation. The biological sample may be a blood, serum or plasma sample, and the assay method may be an immunoassay.

Cross Indications and Longevity

The risk of acquiring a cardiovascular disease increases with age, smoking, hypercholesterolemia (high cholesterol levels), diabetes and hypertension (high blood pressure). It is an object of the present invention to provide means for monitoring and classifying individuals with any of the above indications according to their YKL-40 levels as measured from a biological sample obtained from said individual. The reference level may be any of the YKL-40 levels as described herein, especially as described in the section “reference levels”. Furthermore, YKL-40 may be used as a biomarker for indication of longevity; the lower the level of serum YKL-40 the better prognosis of survival.

An embodiment of the present invention is the method of classifying and/or monitoring one or more individuals according to a prognosis of their survival, said method comprising: measuring the level of YKL-40 in a biological sample from said individual, and comparing the measured level to a reference level of YKL-40. The reference level may be any of the YKL-40 levels as described herein, especially as described in the section “reference levels”. In an additional embodiment, instructions on how to perform the classification and/or monitoration is comprised within a kit of parts along with elements required for the detection and quantification of YKL-40 in biological samples.

YKL-40

YKL-40 is named based on its three N-terminal amino acids Tyrosine (Y), Lysine (K) and Leucine (L) and its molecular mass of about 40 kDa (Johansen et al. 1992). The complete amino acid (SEQ ID NO: 2) and coding sequence (SEQ ID NO: 1) of human YKL-40 is found in GenBank under Accession number: M80927. Human YKL-40 contains a single polypeptide chain of 383 amino acids and is a phylogenetically highly conserved heparin- and chitin-binding plasma glycoprotein. The sequence identity between human YKL-40 and homologs from several other mammals is: pig (84% sequence identity), cow (83%), goat (83%), sheep (83%), guinea pig, rat (80%), and mouse (73%). YKL-40 is a member of “mammalian chitinase-like proteins”, but has no chitinase activity. YKL-40 expression in vitro is absent in normal human monocytes but strongly induced during late stages of macrophage differentiation by activated monocytes and neutrophils, by vascular smooth muscle cells, cancer cells and arthritic chondrocytes. In vivo YKL-40 mRNA and protein are expressed by a subpopulation of macrophages in tissues with inflammation such as atherosclerotic plaques, arthritic vessels of individuals with giant cell arthritis, inflamed synovial membranes, sarcoid lesions, and by peritumoral macrophages.

The molecular processes governing the induction of YKL-40 and its precise functions are unknown. YKL-40 is a secreted protein suggesting that its sites of actions are most likely to be extracellular; however, specific cell-surface or soluble receptors for YKL-40 have not yet been identified. YKL-40 is a growth factor for fibroblasts and chondrocytes, acts synergistically with IGF-1, is regulated by TNF and IL-6, and requires sustained activation of NF-kappaB (Millis et al., 1986) YKL-40 treatment of fibroblasts can counteract the inflammatory response to TNF and IL-1 by phosphorylation of AKT, thereby attenuating ASK1 mediated signaling pathways (Junker et al., 2005; Nøjgaard et al., 2003). This leads to decreased levels of metalloproteinase and IL-8 expression (Junker et al., 2005; Nøjgaard et al., 2003). Furthermore, YKL-40 binds to collagen types I, II and III and modulates the rate of type I collagen fibril formation (Kamal et al., 2006) These observations suggest that YKL-40 may play a protective role in inflammatory environments, limiting degradation of the extracellular matrix and thereby controlling tissue remodeling. YKL-40 also acts as a chemo-attractant for endothelial cells, stimulates their migration and promotes migration and adhesion of vascular smooth muscle cells (Nishikawa et al., 2003; Boot et al., 1999) suggesting a role in angiogenesis. YKL-40 is also a growth factor for fibroblasts, (Vind et al., 2003; Shackelton et al., 1995; Renkema et al., 1998, De Ceunicnck et al., 2001, Recklies et al., 2002, Ling et al., 2004, Recklies et al., 2005) and has an anti-catabolic effect preserving extracellular matrix during tissue remodeling. In addition, macrophages in atherosclerotic plaques express YKL-40 mRNA, particularly macrophages that have infiltrated deeper in the lesion, and the highest YKL-40 expression is found in macrophages in the early lesion of atherosclerosis (Boot et al., 1999). Furthermore YKL-40 can be regarded as an acute phase protein, since its plasma or serum concentration is increased in several inflammatory diseases.

Cellular receptors mediating the biological effects of YKL-40 are not known, but the activation of cytoplasmic signal-transduction pathways suggests that YKL-40 interacts with signaling components on the cell membrane.

It is an object of the present invention to detect any transcriptional product of the YKL-40 gene. A transcriptional product of the gene may thus be hnRNA, mRNA, full length protein, fragmented protein, or peptides of the YKL-40 protein. It is understood that one or more proteins, RNA transcripts, fragments and/or peptides may be detected simultaneously. It is furthermore an aspect of the present invention to detect transcriptional products by any means available such as by immunoassays such as antibody detection of the YKL-40 protein, fragments or peptides hereof, as well as by detection by PCR based assays such as detection of RNA by RT-PCR.

YKL-40 and Heart Disease

The present invention discloses a method of classifying and/or monitoring individuals according to a prognosis of their survival based on the measurement of YKL-40 levels in biological samples and comparing the found values with one or more reference levels. The individuals may be healthy individuals or individuals suffering from any of the above mentioned heart diseases. Of particular interest to the present invention are heart diseases or disorders caused by atherosclerosis.

As can be ascertained from the below and from the results given in the Examples, YKL-40 levels are increased in individuals suffering from heart diseases. As can also be seen from these results, the higher the level of YKL-40, the shorter is the prognosis for the survival of the individual.

YKL-40 has therefore surprisingly been found to be a new biomarker, the level of which gives a prognosis for the survival of the individual.

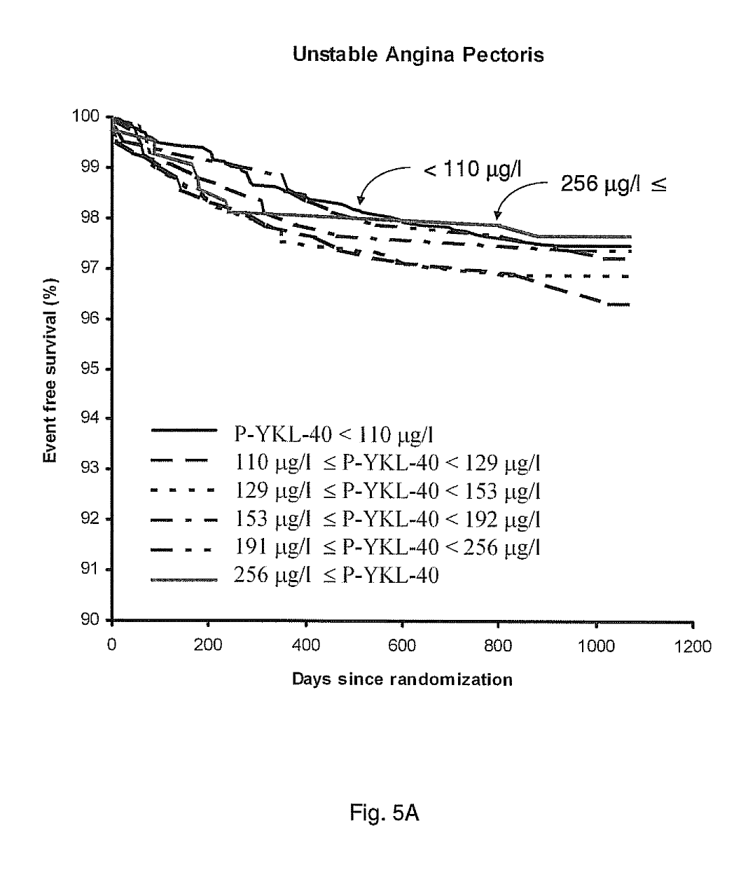

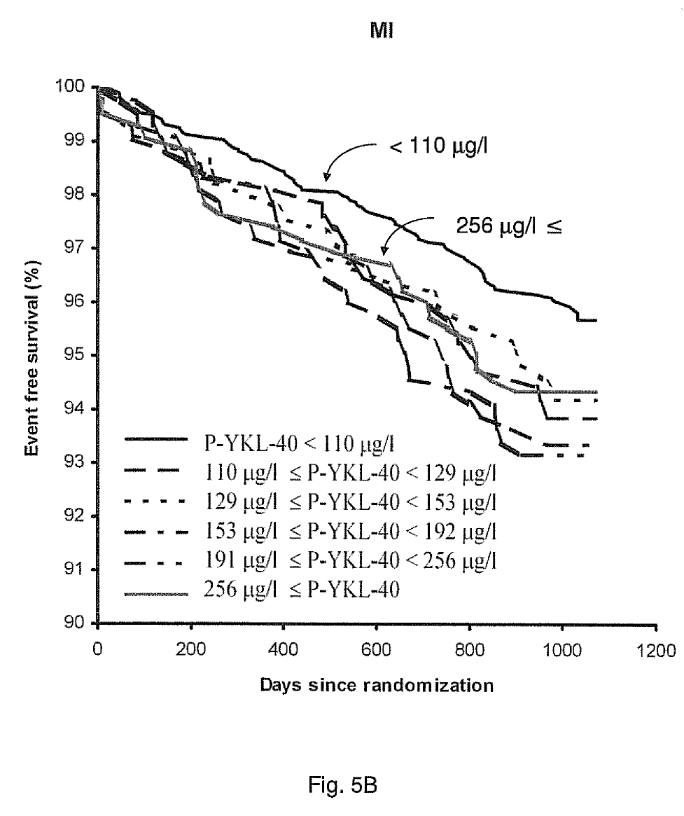

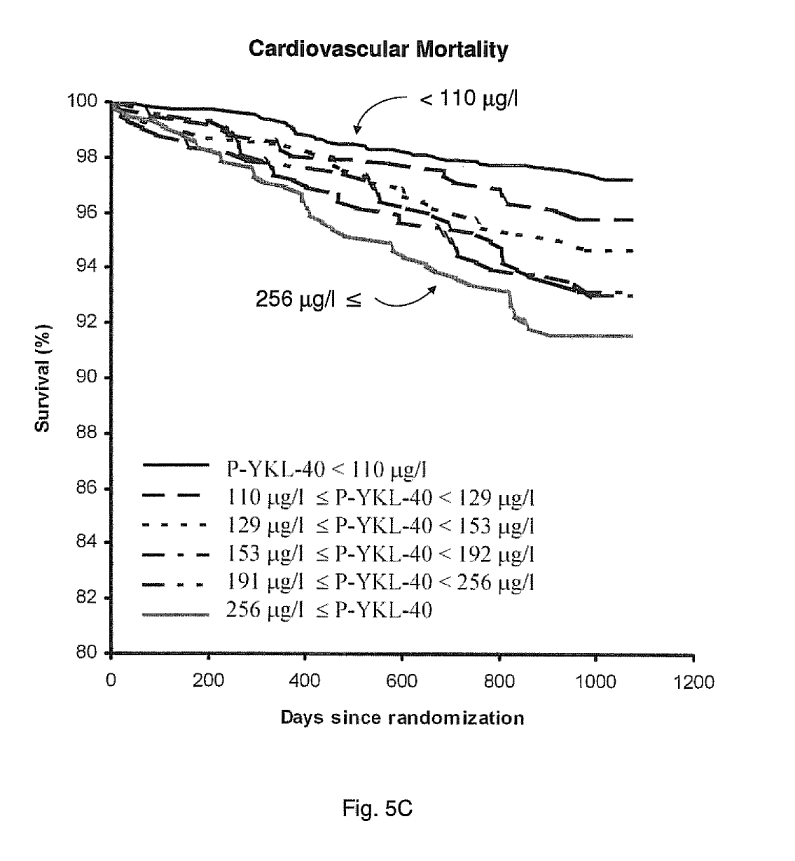

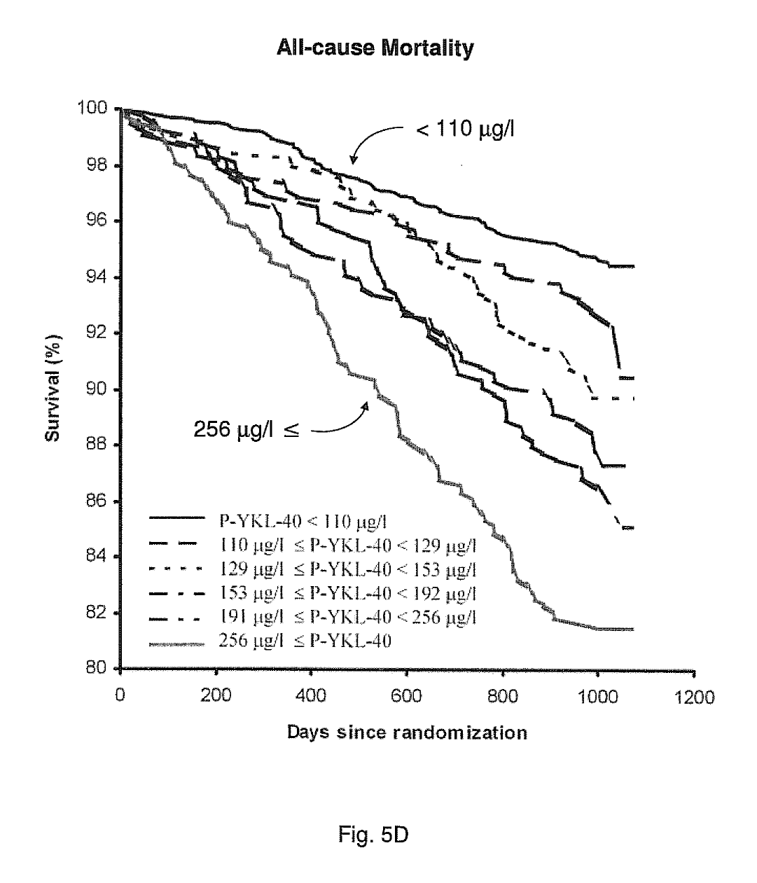

Serum YKL-40 levels are increased in individuals with chronic coronary artery disease compared to controls as described in the Examples. Therefore, YKL-40 is a new biomarker of changes in chronic myocardial ischemia and/or angiogenesis in individuals with coronary artery disease and acts as a prognostic marker of survival, or as a prognostic marker for new events of e.g. myocardial infarction. As can be ascertained from the examples, there is a clear reduction in event-free survival for cardiovascular death with increasing serum YKL-40. The cardiovascular mortality was 8.0% for the highest serum YKL-40 (Group VI, serum YKL-40≧256 μg/l) and 2.6% for low serum YKL-40 (Group I, serum YKL-40<110 μg/l) (FIG. 5C). This is a more than 3 fold difference in cardiovascular mortality rate between individuals classified according to YKL-40 levels being high or low. This association is even clearer for all-cause mortality (FIG. 5D). 18.4% of the patients with the highest serum YKL-40 died within 2.6 years compared to 5.3% of the patients with low serum YKL-40, that is a factor of almost 3.5.

A method of classifying and/or monitoring individuals based on the prognosis of their survival as found by measuring YKL-40 levels in samples taken from these individuals is an object of the present invention. Specifically, it is an object of the present invention to provide a method for classifying and/or monitoring individuals who suffer from coronary artery disease (CAD) according to a prognosis of the survival of said individuals. More specifically, the method relates to individuals who suffer from CAD affecting at least one vessel, such as two vessels, such as three vessels, such as four vessels, such as five vessels, or such as six vessels. It is furthermore an aspect of the present invention that the affected vessels may be end vessels or collateral vessels or, if two or more vessels are affected, that it may be a combination of end and collateral vessels, that are affected.

The extent of CAD cannot be judged from the number of affected vessels. Rather the extent of CAD relates to the extent of atherosclerosis present in the affected vessels in the individual. Likewise, an individual suffering from CAD wherein multiple vessels are affected may have fewer symptoms than an individual having a single affected vessel.

An increased YKL-40 level is also a prognostic biomarker for myocardial infarction, as can be seen in the Examples. It has surprisingly been found that YKL-40 is significantly associated with both MI and cardiovascular death. It has previously been established that MI is the main cause of cardiovascular death. Therefore it is an object of the present invention to provide a method for classifying and/or monitoring individuals who suffer from myocardial infarction (MI) according to a prognosis of the survival of said individuals.

Serum YKL-40 levels are increased the day after an acute ST-elevation myocardial infarction (STEMI) compared to controls as described in the Examples. Therefore, YKL-40 is a new biomarker of changes in chronic myocardial ischemia and/or angiogenesis in individuals with coronary artery disease and acts as a prognostic marker of survival. It is thus an object of the present invention to provide a method for classifying and/or monitoring individuals who suffer from acute coronary syndrome according to a prognosis of the survival of said individuals. Specifically, it is an object of the present invention to provide a method of classifying and/or monitoring individuals suffering from either non-ST segment elevation myocardial infarction (NSTEMI) or ST segment elevation myocardial infarction (STEMI) either of these being fatal or non-fatal.

In the Examples, it is described how, during a relatively short follow-up period of 2.6 years, the HRs for serum YKL-40 are high, 1.83 for MI (acute myocardial infarction), 3.28 for cardiovascular death and 3.75 for all-cause mortality. These HR values are high; an indication of the strength YKL-40 has as a biomarker.

Detection of YKL-40

Peptides and polynucleotides of the invention include functional derivatives of YKL-40, YKL-40 peptides and nucleotides encoding therefore. By “functional derivative” is meant the “fragments,” “variants,” “analogs,” or “chemical derivatives” of a molecule. A “fragment” of a molecule, such as any of the DNA sequences of the present invention, includes any nucleotide subset of the molecule. A “variant” of such molecule refers to a naturally occurring molecule substantially similar to either the entire molecule, or a fragment thereof. An “analog” of a molecule refers to a non-natural molecule substantially similar to either the entire molecule or a fragment thereof.

A molecule is said to be “substantially similar” to another molecule if the sequence of amino acids in both molecules is substantially the same. Substantially similar amino acid molecules will possess a similar biological activity. Thus, provided that two molecules possess a similar activity, they are considered variants as that term is used herein even if one of the molecules contains additional amino acid residues not found in the other, or if the sequence of amino acid residues is not identical.

Further, a molecule is said to be a “chemical derivative” of another molecule when it contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, for example, in Remington's Pharmaceutical Sciences, 16th Ed., Mack Publishing Co., Easton, Pa., 1980.

Minor modifications of the YKL-40 primary amino acid sequence may result in proteins and peptides that have substantially similar activity as compared to the YKL-40 peptides described herein. Such modifications may be deliberate, as by site-directed mutagenesis, or may be spontaneous. All of the peptides produced by these modifications are included herein as long as the biological activity of YKL-40 still exists. Further, deletion of one or more amino acids can also result in a modification of the structure of the resultant molecule without significantly altering its biological activity. This can lead to the development of a smaller active molecule which would have broader utility. For example, one can remove amino or carboxy terminal amino acids which may not be required for the enzyme to exert the desired catalytic or antigenic activity.

Either polyclonal or monoclonal antibodies may be used in the immunoassays and therapeutic methods of the invention described below. Some anti-YKL-40 antibodies are available commercially or may alternatively be raised as herein described or known in the art. Polyclonal antibodies may be raised by multiple subcutaneous or intramuscular injections of substantially pure YKL-40 or antigenic YKL-40 peptides into a suitable non-human mammal. The antigenicity of YKL-40 peptides can be determined by conventional techniques to determine the magnitude of the antibody response of an animal which has been immunized with the peptide. Generally, the YKL-40 peptides which are used to raise the anti-YKL-40 antibodies should generally be those which induce production of high titers of antibody with relatively high affinity for YKL-40.

If desired, the immunizing peptide may be coupled to a carrier protein by conjugation using techniques which are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit). Because YKL-40 may be conserved among mammalian species, use of a carrier protein to enhance the immunogenicity of YKL-40 proteins is preferred.

The antibodies are then obtained from blood samples taken from the mammal. The techniques used to develop polyclonal antibodies are known in the art see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies, see, for example, Coligan, et al., Unit 9, Current Protocols in Immunology, Wiley Interscience, 1991).

Preferably, however, the YKL-40 antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′).sub.2, which are capable of binding an epitopic determinant. Also, in this context, the term “mAb's of the invention” refers to monoclonal antibodies with specificity for YKL-40.

The general method used for production of hybridomas secreting mAbs is well known (Kohler and Milstein, 1975). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines.

Confirmation of YKL-40 specificity among mAb's can be accomplished using relatively routine screening techniques (such as the enzyme-linked immunosorbent assay, or “ELISA”) to determine the elementary reaction pattern of the mAb of interest. It is also possible to evaluate an mAb to determine whether it has the same specificity as a mAb of the invention without undue experimentation by determining whether the mAb being tested prevents a mAb of the invention from binding to YKL-40 isolated as described above, if the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to pre-incubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

Immunoassay Procedures

The immunoassay procedure used must be quantitative so that levels of YKL-40 in a individual with disease may be distinguished from normal levels which may be present in healthy humans and/or background levels measured in the individual. Competitive and sandwich assays on a solid phase using detectible labels (direct or indirect) are, therefore, preferred. The label will provide a detectible signal indicative of binding of antibody to the YKL-40 antigen. The antibody or antigen may be labeled with any label known in the art to provide a detectible signal, including radioisotopes, enzymes, fluorescent molecules, chemiluminescent molecules, bioluminescent molecules and colloidal gold. Of the known assay procedures, radioimmunoassay (RIA) is most preferred for its sensitivity. A radioisotope will, therefore, be the preferred label.

Examples of metallic ions which can be directly bound to an antibody, or indirectly bound to the YKL-40 antigen are well-known to those of ordinary skill in the art and include .sup.125 I, .sup.111 In, .sup.97 Ru, .sup.67 Ga, .sup.68 Ga, .sup.72 As, .sup.89 Zr, .sup.90 Y and .sup.201 TI. Preferred for its ease of attachment without compromise of antigen binding specificity is .sup.125 I (sodium salt, Amersham, United Kingdom). Labeling of YKL-40 with .sup.125 I may be performed according to the method described in Salacinski, et al. (1981). Iodogen for use to provide the .sup.125 I label (1,3,4,6-tetrachloro-3.alpha., 6.alpha.-diphenyl glycoluril) is commercially available from Pierce and Warriner, Chester, England.

The radioimmunoassay of the invention uses standards or samples incubated with a substantially equal volume of YKL-40 antiserum and of YKL-40 tracer. Standards and samples are generally assayed in duplicate. The sensitivity (detection limit) of the assay of the invention is about 10 μg/l. Sensitivity in this context is defined as the detectible mass equivalent to twice the standard deviation of the zero binding values. The standard curve will generally be linear between 20 and 100 μg/l The intra- and interassay coefficients of variance for the assay described in the following examples are <6.5% and <12%, respectively.

It will be appreciated by those skilled in the art that, although not necessarily as sensitive as an RIA, assay procedures using labels other than radioisotopes have certain advantages and may, therefore, be employed as alternatives to the preferred RIA format. For example, an enzyme-linked immunosorbent assay (ELISA) may be readily automated using an ELISA microtiter plate reader and reagents which are readily available in many research and clinical laboratories. Fluorescent, chemiluminescent and bioluminescent labels have the advantage of being visually detectible, though they are not as useful as radioisotopes to quantify the amount of antigen bound by antibody in the assay.

PCR Based Assays

Further, it will be appreciated by those of skill in the art that means other than immunoassays may be employed to detect and quantify the presence of YKL-40 in a biological sample. For example, a polynucleotide encoding YKL-40 may be detected using quantitative polymerase chain reaction (PCR) protocols known in the art. The preferred method for performance of quantitative PCR is a competitive PCR technique performed using a competitor template containing an induced mutation of one or more base pairs which results in the competitor differing in sequence or size from the target YKL-40 gene template. One of the primers is biotinylated or, preferably, aminated so that one strand (usually the antisense strand) of the resulting PCR product can be immobilized via an amino-carboxyl, amino—amino, biotin-streptavidin or other suitably tight bond to a solid phase support which has been tightly bound to an appropriate reactant. Most preferably, the bonds between the PCR product, solid phase support and reactant will be covalent ones, thus reliably rendering the bonds resistant to uncoupling under denaturing conditions.

Once the aminated or biotinylated strands of the PCR products are immobilized, the unbound complementary strands are separated in an alkaline denaturing wash and removed from the reaction environment. Sequence-specific oligonucleotides (“SSO's”) corresponding to the target and competitor nucleic acids are labelled with a detection tag. The SSO's are then hybridized to the antisense strands in absence of competition from the removed unbound sense strands. Appropriate assay reagents are added and the degree of hybridization is measured by ELISA measurement means appropriate to the detection tag and solid phase support means used, preferably an ELISA microplate reader. The measured values are compared to derive target nucleic acid content, using a standard curve separately derived from PCR reactions amplifying templates including target and competitor templates. This method is advantageous in that it is quantitative, does not depend upon the number of PCR cycles, and is not influenced by competition between the SSO probe and the complementary strand in the PCR product.

Alternatively, part of the polymerization step and the entire hybridization step can be performed on a solid phase support. In this method, it is a nucleotide polymerization primer (preferably an oligonucleotide) which is captured onto a solid phase support rather than a strand of the PCR products. Target and competitor nucleic acid PCR products are then added in solution to the solid phase support and a polymerization step is performed. The unbound sense strands of the polymerization product are removed under the denaturing conditions described above.

A target to competitor nucleic acid ratio can be determined by detection of labeled oligonucleotide SSO probes using appropriate measurement means (preferably ELISA readers) and standard curve as described supra. The efficiency of this method can be so great that a chain reaction in the polymerization step may be unnecessary, thus shortening the time needed to perform the method. The accuracy of the method is also enhanced because the final polymerization products do not have to be transferred from a reaction tube to a solid phase support for hybridization, thus limiting the potential for their loss or damage. If necessary for a particular sample, however, the PCR may be used to amplify the target and competitor nucleic acids in a separate reaction tube, followed by a final polymerization performed on the solid phase support.

Molecules capable of providing different, detectible signals indicative of the formation of bound PCR products known to those skilled in the art (such as labeled nucleotide chromophores which will form different colors indicative of the formation of target and competitor PCR products) can be added to the reaction solution during the last few cycles of the reaction. The ratio between the target and competitor nucleic acids can also be determined by ELISA or other appropriate measurement means and reagents reactive with detection tags coupled to the 3′ end of the immobilized hybridization primers. This method may also be adapted to detect whether a particular gene is present in the sample (without quantifying it) by performing a conventional noncompetitive PCR protocol.

Those of ordinary skill in the art will know, or may readily ascertain, how to select suitable primers for use in the above methods. For further details regarding the above-described techniques, reference may be made to the disclosures in Kohsaka, et al., Nuc. Acids Res., 21:3469-3472, 1993; Bunn, et al., U.S. Pat. No. 5,213,961; and to Innis, et al., PCR Protocols: A Guide to Methods and Applications, Acad. Press, 1990, the disclosures of which are incorporated herein solely for purposes of illustrating the state of the art regarding quantitative PCR protocols.

Enzymatic Assays

YKL-40 appears to be a hydrolytic enzyme and therefore the level of functional YKL-40 protein or protein fragments may be determined based on an enzymatic assay in which the substrate of YKL-40 is hydrolyzed into a detectable form.

Dipstick

A particular method of detecting YKL-40 relates to a device comprising a rapid, qualitative and/or quantitative test system mounted on a solid support for the determination of YKL-40 levels in biological samples.

The test system may make use of any of the above mentioned assay systems, such as an immunoassay, a PCR based assay or an enzymatic assay. An immunoassay is preferred for the present test system.

The solid support can be a any phase used in performing any of the above assays, particularly immunoassays, including dipsticks, membranes, absorptive pads, beads, microtiter wells, test tubes, and the like. Preferred are test devices which may be conveniently used by the testing personnel or the patient for self-testing, having minimal or no previous training. Such preferred test devices include dipsticks and membrane assay systems. The preparation and use of such conventional test systems is well described in the patent, medical, and scientific literature. If a stick is used, the anti-YKL-40 antibody is bound to one end of the stick such that the end with the antibody can be dipped into or onto the biological samples. Alternatively, the samples can be applied onto the antibody-coated dipstick or membrane by pipette, dropper, tweezers or the like, or be squirted directly from the body and onto the stick.

In the present embodiment any biological sample that is or may be converted to a fluid is preferred. Particularly biological samples that are obtainable from a body as a fluid are preferred; examples hereof include, and are not limited to: blood, serum, plasma, urine, cerebral fluid, joint fluid, semen, and saliva.

The antibody against YKL-40 can be of any isotype, such as IgA, IgG or IgM, Fab fragments, or the like. The antibody may be a monoclonal or polyclonal and produced by methods as generally described in Harlow and Lane, Antibodies, A Laboratory Manual, Cold Spring Harbor Laboratory, 1988, incorporated herein by reference. See also section on immunoassays. The antibody can be applied to the solid support by direct or indirect means. Indirect bonding allows maximum exposure of the YKL-40 binding sites to the assay solutions since the sites are not themselves used for binding to the support. Polyclonal antibodies may be used since polyclonal antibodies can recognize different epitopes of YKL-40 thereby enhancing the sensitivity of the assay. Alternatively, monoclonal antibodies against YKL-40 may be used.

The solid support is preferably non-specifically blocked after binding the YKL-40 antibodies to the solid support. Non-specific blocking of surrounding areas can be with whole or derivatized bovine serum albumin, or albumin from other animals, whole animal serum, casein, non-fat milk, and the like.

The sample is applied onto the solid support with bound YKL-40-specific antibody such that the YKL-40 will be bound to the solid support through said antibodies. Excess and unbound components of the sample are removed and the solid support is preferably washed so the antibody-antigen complexes are retained on the solid support. The solid support may be washed with a washing solution which may contain a detergent such as Tween-20, Tween-80 or sodium dodecyl sulphate.

After the YKL-40 has been allowed to bind to the solid support, a second antibody which reacts with YKL-40 is applied. The second antibody may be labelled, preferably with a visible label. The labels may be soluble or particulate and may include dyed immunoglobulin binding substances, simple dyes or dye polymers, dyed latex beads, dye-containing liposomes, dyed cells or organisms, or metallic, organic, inorganic, or dye solids. The labels may be bound to the YKL-40 antibodies by a variety of means that are well known in the art. In some embodiments of the present invention, the labels may be enzymes that can be coupled to a signal producing system. Examples of visible labels include alkaline phosphatase, beta-galactosidase, horseradish peroxidase, and biotin. Many enzyme-chromogen or enzyme-substrate-chromogen combinations are known and used for enzyme-linked assays.

Simultaneously with the sample, corresponding steps may be carried out with a known amount or amounts of YKL-40 and such a step can be the standard for the assay.

The solid support is washed again to remove unbound labelled antibody and the labeled antibody is visualized and quantitated. The accumulation of label will generally be assessed visually. This visual detection may allow for detection of different colors, e.g., red color, yellow color, brown color, or green color, depending on label used. Accumulated label may also be detected by optical detection devices such as reflectance analyzers, video image analyzers and the like. The visible intensity of accumulated label could correlate with the concentration of YKL-40 in the sample. The correlation between the visible intensity of accumulated label and the amount of YKL-40 may be made by comparison of the visible intensity to a set of reference standards. Preferably, the standards have been assayed in the same way as the unknown sample, and more preferably alongside the sample, either on the same or on a different solid support. The concentration of standards to be used can range from about 1 μg of YKL-40 per liter of solution, up to about 1 mg of YKL-40 per liter of solution, preferably the range for testing serum samples will be from 50 μg/l to 400 μg/l YKL-40. Preferably, several different concentrations of YKL-40 standards are used so that quantitating the unknown by comparison of intensity of color is more accurate. An intensity of color similar to 110 μg/l of YKL-40 is considered negative, as compared with an intensity of color similar to 200 μg/l.

The device, such as the herein described dipstick or other solid support based test system, may thus be used in aid of determining the approximate level of YKL-40 in a biological sample by comparison to one or more standards/control fields. Thus the concentration of YKL-40 can be ascertained to be within a range between two of the concentrations of YKL-40 applied to the standard/control fields of the device. Alternatively the concentration of YKL-40 can be judged to be above or below a cut-off value of YKL-40, the chosen concentration for the cut-off value being applied to the control field of the dipstick.

It is an object of the present invention to provide a device for the classification and/or monitoration of individuals based on a prognosis of their survival as found by comparing the level of YKL-40 in a biological sample from said individual, with that of a reference level of YKL-40. There may be multiple reference levels/standards available within and/or on the device or single reference level/standard within and/or on the device. In the latter case, the device is for the classification and/or monitoration of individuals based on a prognosis of their survival as found by comparing the level of YKL-40 in a biological sample from said individual, with that of a cut-off value of YKL-40. Any cut off value (concentration) of YKL-40 may be used as discussed herein above.

A specific embodiment of the device according to the present invention relates to a device comprising means for measuring the level of YKL-40 in a sample; and means for comparing the measured level of YKL-40 with at least one reference level of YKL-40. The reference level may preferably be a single reference level such as e.g. the cut-off value of about 80 μg/l, or alternatively the reference level may a set of cut-off values of YKL-40. If this is the case the device comprises means for comparing the measured level of YKL-40 with at a set of cut-off values for YKL-40.

Additionally, the assay can be used to monitor the YKL-40 levels of a patient during therapy since YKL-40 levels should decrease if the therapy is useful. As evident to a person with ordinary skill in the art, it may be necessary to undergo one or more serial dilutions of the patients sample such that the level of YKL-40 in the patients sample can be compared to one of the set standards. The patient YKL-40 measurement is then corrected for the dilution factor.

Although each of the steps can be carried out in the same vessel, such as a test tube, if it is cleaned and washed after each of the steps, a fast and convenient on-site assay is best performed according to the invention by using three separate vessels for each of the steps, one for the sample, one for washing, and one for developing the detectable label.

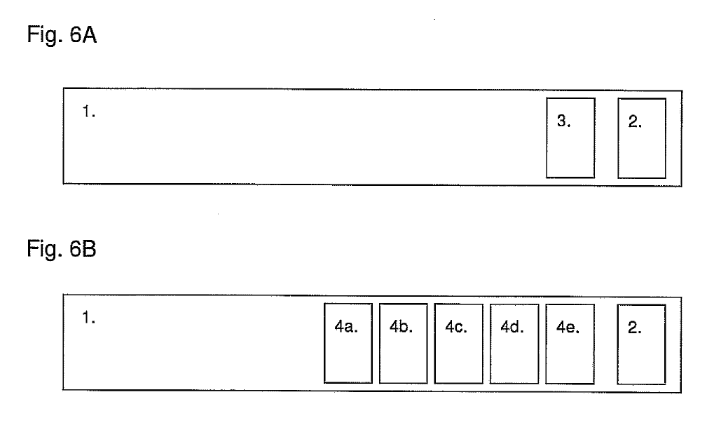

It is thus an object of the present invention that the YKL-40 level of a biological sample for use in the classification according to a reference level of YKL-4 of the individual from which the biological sample originated is measured by use of a dipstick. (FIGS. 6A and 6B)

All the materials and reagents required for assaying YKL-40 according to the present invention can be assembled together in a kit. This generally will comprise one or more solutions containing a known concentration of YKL-40, a washing solution, a solution of a chromogen which changes color or shade by the action of the enzyme directly or indirectly through action on a substrate, an anti-YKL-40 antibody conjugated to a label such that it could be detected, pipettes for the transfer of said solutions, test tubes for said solutions, and a solid support, in particular adapted to be inserted into the test tubes, carrying on the surface thereof a polyclonal antibody to YKL-40. The kit may also contain one or more solid support having an anti-YKL-40 antibody for use in assaying one or more samples simultaneously or individually, and the necessary reagent required to develop the label. It is also preferable that the YKL-40 used for standards be provided so that it could be assayed fresh along with the unknown sample. Such kits will comprise distinct containers for each individual reagent.

In the above test kit, the reagents may be supplied from storage bottles or one or more of the test tubes may be prefilled with the reagents or controls.

The components of the kit may also be provided in dried or lyophilized forms. When reagents or components are provided as a dried form, reconstitution generally is by the addition of a suitable solvent. It is envisioned that the solvent also may be provided in another container means.

The kits of the present invention also will typically include a means for containing the reagents such as vials or tubes in close confinement for commercial sale such as, e.g. injection or blow-molded plastic containers into which the desired vials are retained. The kits will also comprise a set of instructions on how to perform the assay.

In an alternative embodiment, the dipstick and/or kit will comprise means for assaying other biomarkers than YKL-40, such as any one or more of the biomarkers from the following non-limiting group: C-reactive protein (CRP), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases, pentraxin 3, secretory phospholipase A2 group IIA, intercellular adhesion molecule-1, Heart-type fatty acid-binding protein (H-FABP), Myosin light chain-1 (MLC-1), P-selectin and CKMB. Preferably the dipstick and/or kit will comprise means for assaying C-reactive protein and/or brain natriuretic protein and/or homocysteine.

Other Biomarkers

YKL-40 is an independent biomarker for all death and cardiac death and may be used accordingly. However, YKL-40 may also be used in combination with other known biomarkers such as C-reactive protein (CRP), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases, pentraxin 3, secretory phospholipase A2 group IIA, intercellular adhesion molecule-1, Heart-type fatty acid-binding protein (H-FABP), Myosin light chain-1 (MLC-1), P-selectin and CKMB. Of the mentioned biomarkers, both the soluble and insoluble forms of the proteins are of relevance for the present invention, such as UPAR and soluble UPAR; intercellular adhesion molecule-1 and soluble intercellular adhesion molecule-1 and others. The levels of any of the abovementioned markers may be measured in a biological sample such as a blood, serum, plasma or tissue sample and by any means available such as by use of immunoassays or PCR based assays or several assay types in combination.

It is thus an aspect of the present invention to provide means for classifying and monitoring individuals according to their YKL-40 levels in combination with levels of other biomarkers these being selected from the non-limiting group consisting of C-reactive protein (CRP), brain natriuretic protein (BNP), interleukins and tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB. Of these additional biomarkers C-reactive protein, brain natriuretic protein and homocysteine are of particular interest.

In a preferred embodiment of the methods according to the invention the YKL-40 level is measured together with the level of another biomarker. Preferably the other biomarker is selected from the group consisting of C-reactive protein (CRP), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Myosin light chain-1 (MLC-1), P-selectin and CKMB.

The abovementioned embodiment may be comprised in a kit of parts together with any required medical and or sampling equipment and instructions for use of the equipment and how to perform the assay of choice.

Biological Sample

A biological sample is a sample obtained from an individual. As such a biological sample may be a sample selected from the group consisting of tissue, bone, blood, serum and plasma samples. Of special relevance to the present invention are samples of blood, serum and plasma. Those of ordinary skill in the art will be able to readily determine which assay sample source is the most appropriate for use in the classification or monitoration of a particular disease, or disorder or general state of health for any of which an increased YKL-40 level is prognostic of survival.

Individual

The individuals herein referred to are single members of a species, herein preferably a mammalian species. Any mammalian species is an object of the present invention, although any of the following species are of particular relevance: mouse, rat, guinea pig, hamster, rabbit, cat, dog, pig, cow, horse, sheep, monkey, and human. Most preferably the individual of the present invention is a human. The individuals may in the present text also be referred to as patients or subjects.

Kit of Parts

Another embodiment of the present invention comprises a kit of parts, wherein the kit includes at least elements in aid of assessing the level of YKL-40 in a biological sample obtained from an individual, and the instruction on how to do so. Said elements may be a method of detecting the YKL-40 levels such as an immunoassay, or parts required to perform an immunoassay specific for YKL-40 detection. Optionally, a kit may further or alternatively comprise elements for performing PCR based assays for the detection of YKL-40 and determination of levels of the same from biological samples. The kit of parts may further comprise equipment for obtaining one or more biological samples, such equipment may for example be syringes, vials or other. The kit of parts may be packed for single use or for repeated usage, and the elements therein may be disposable such as to be disposed of after a single use or may be of a quality that allows repeated usage.

A specific embodiment of a kit of parts according to the present invention relates to a kit of parts comprising means for detecting YKL-40 in a biological sample; means for comparing the measured level of YKL-40 with a reference level of YKL-40; and instruction on how to classify and/or monitor individuals according to their YKL-40 levels.

In one embodiment the kit of parts, apart from being for the detection and determination of YKL-40 levels, may further comprise meansfor detecting one or more additional biomarkers such as C-reactive protein, homocysteine, brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), and CKMB. Preferably, such a kit of parts that includes the detection of other biomarkers than YKL-40 comprises elements for the detection and/or determination of the levels of C-reactive protein, brain natriuretic protein and/or homocysteine in a biological sample.

The kit of parts according to the present invention may furthermore comprise at least one device as described herein above.

### DETAILED DESCRIPTION OF THE DRAWINGS

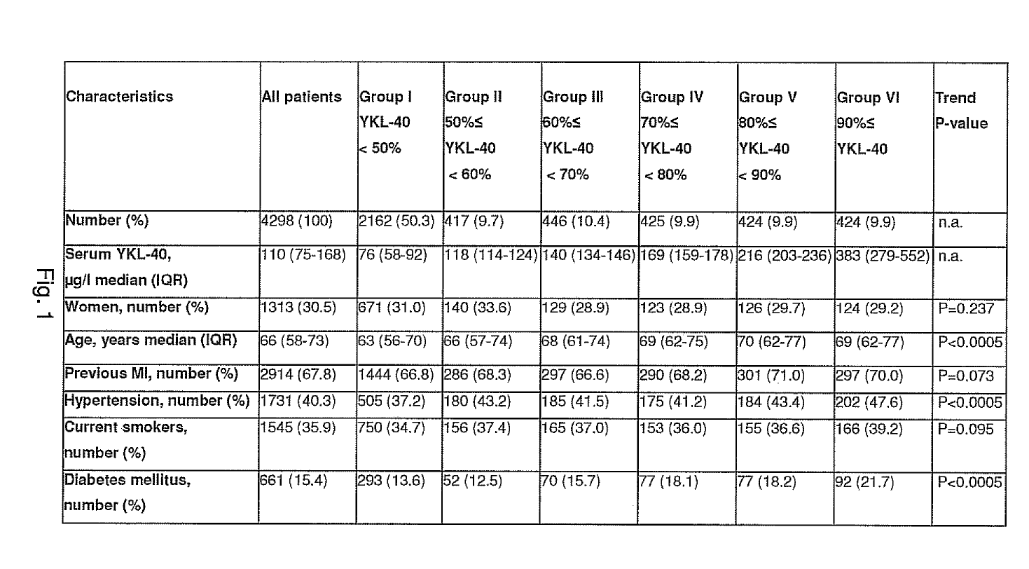

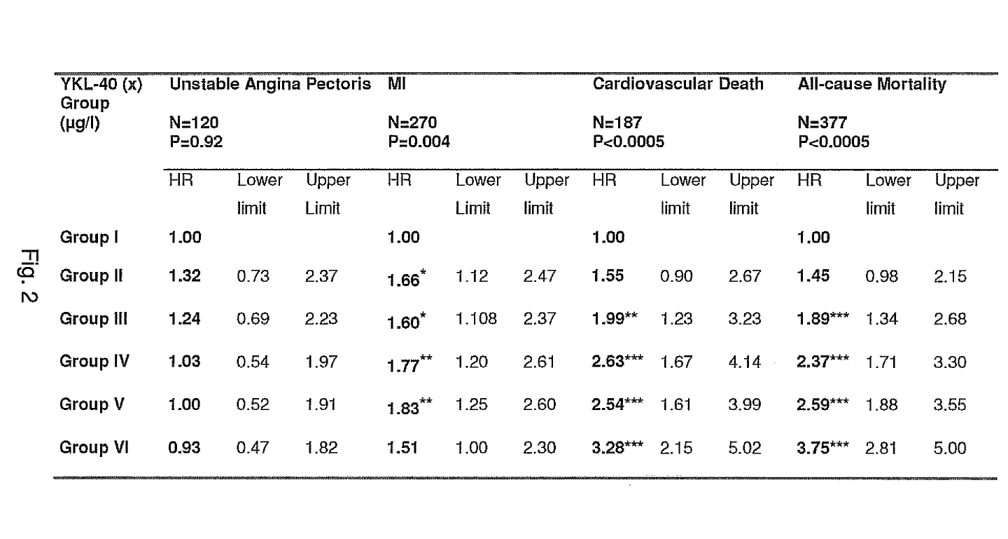

FIG. 1 Demographic data of individuals in the YKL-40 study.
Demographic characteristics of the patients at entry and according to increasing serum concentrations of YKL-40. Groups by serum YKL-40 percentile. Values are expressed as number (percent) or median (IQR, inter-quartile range). Statistical comparisons between the six groups were made using tests for trend. MI, acute myocardial infarction. n.a. not available. Group I: YKL-40<110 μg/l; II: 110 μg/l≦YKL-40<129 μg/l; III: 129 μg/l≦YKL-40<153 μg/l; IV: 153 μg/l≦YKL-40<191 μg/l; V: 191 μg/l≦YKL-40<256 μg/l; and VI: YKL-40: 256 μg/l≦YKL-40.

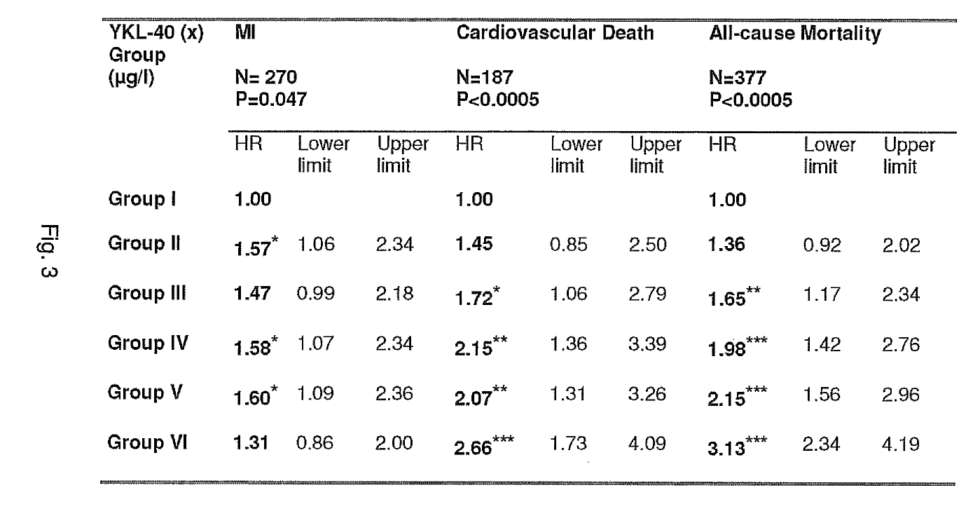

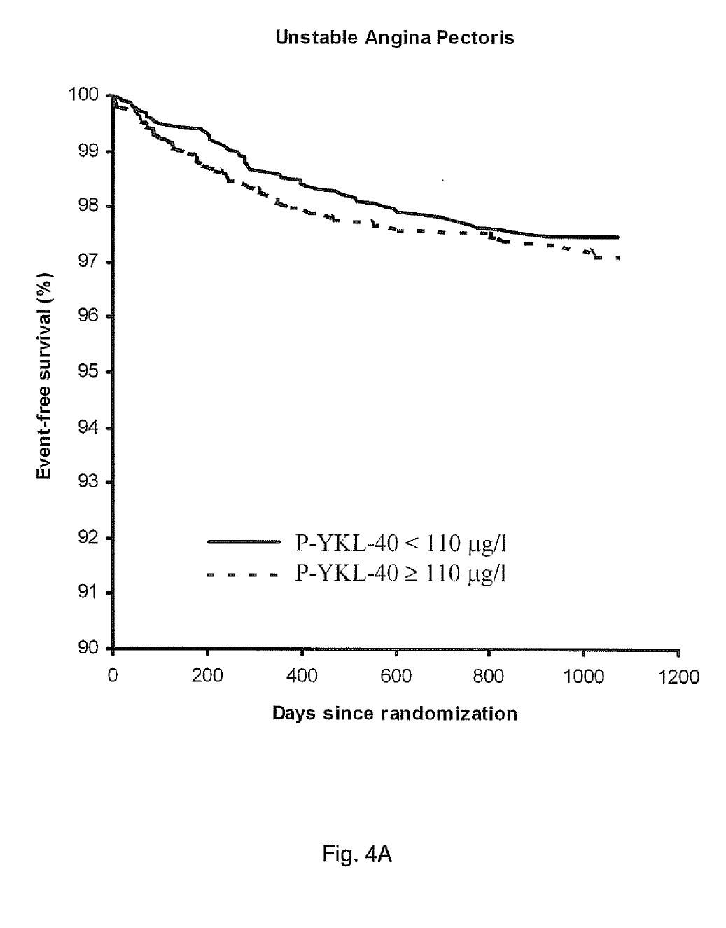

FIG. 2 Hazard ratios.
The hazard ratios (HRs) with 95% confidence limits of each of six serum YKL-40 groups as obtained from four Cox analyses of time to event (unstable angina pectoris, acute myocardial infarction (MI), cardiovascular death, and all-cause mortality) during 2.6 years of follow-up. The intervention indicator (clarithromycin or placebo) was included as a co-variate. P values: * 0.01<P<0.05; ** 0.0005≦P<0.01; and ***P<0.0005. Group I<50% percentile: YKL-40<110 μg/l; II 50%≦YKL-40<60% percentile: 110 μg/l≦YKL-40<129 μg/l; III 60%≦YKL-40<70% percentile: 129 μg/l≦YKL-40<153 μg/l; IV 70%≦YKL-40<80% percentile: 153 μg/l≦YKL-40<191 μg/l; V 80%≦YKL-40<90% percentile: 191 μg/l≦YKL-40<256 μg/l; and VI≧90% percentile YKL-40: 256 μg/l≦YKL-40.

FIG. 3 Hazard ratios including intervention indicator and risk factors.
The hazard ratios (HRs) with 95% confidence limits of each of six serum YKL-40 groups as obtained from three Cox analyses of time to myocardial infarction (MI), cardiovascular death, and all-cause mortality during 2.6 years of follow-up in a multivariate model. The intervention indicator (clarithromycin or placebo) and other risk factors (sex, previous acute myocardial infarction, age (<60; ≧60 years), smoking status, hypertension, and diabetes mellitus) were included as co-variates. P values: * 0.01<P<0.05; ** 0.0005≦P<0.01; and ***P<0.0005. Group I<50% percentile: YKL-40<110 μg/l; II 50%≦YKL-40<60% percentile: 110 μg/l≦YKL-40<129 μg/l; III 60%≦YKL-40<70% percentile: 129 μg/l≦YKL-40<153 μg/l; IV 70%≦YKL-40<80% percentile: 153 μg/l≦YKL-40<191 μg/l; V 80%≦YKL-40<90% percentile: 191 μg/l≦YKL-40<256 μg/l; and VI≧90% percentile YKL-40: 256 μg/l≦YKL-40.

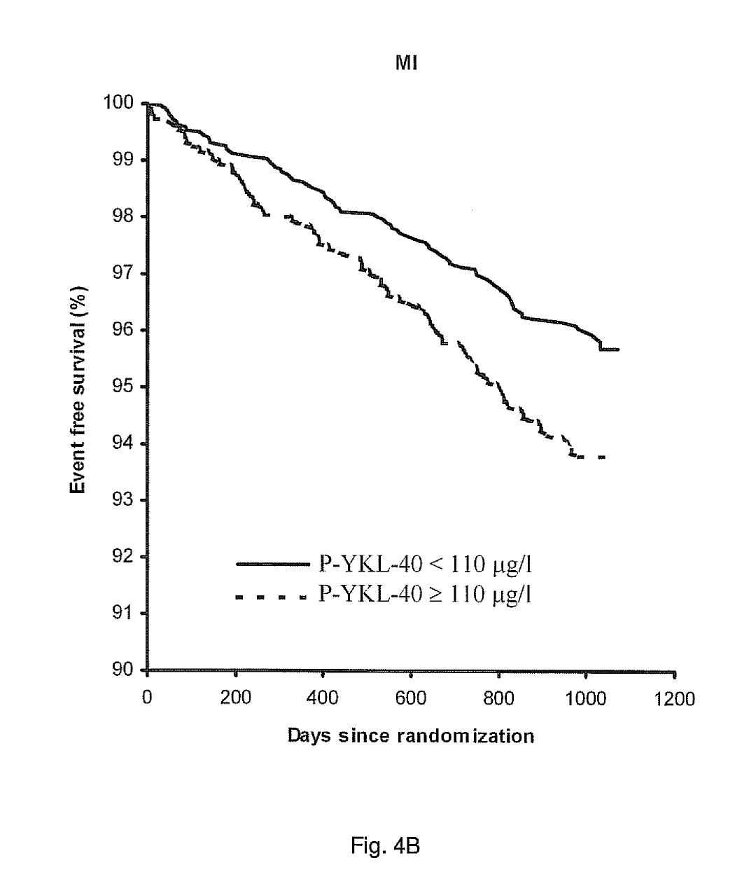

FIG. 4 Survival curve according cut-off value classification.
Event free survival for unstable AP (FIG. 4A) and MI (FIG. 4B) during 2.6 years of follow-up in patients with YKL-40 values above or below 110 μg/l.

FIG. 5 Survival curve according to classification.
Event free survival during 2.6 years of follow-up of each of the six serum YKL-40 groups:
I: <50% percentile: YKL-40<110 μg/l;
II: 50%≦YKL-40<60% percentile: 110 μg/l≦YKL-40<129 μg/l;
III: 60%≦YKL-40<70% percentile: 129 μg/l≦YKL-40<153 μg/l;
IV: 70%≦YKL-40<80% percentile: 153 μg/l≦YKL-40<191 μg/l;
V: ≧80%≦YKL-40<90% percentile: 191 μg/l≦YKL-40<256 μg/l; and
VI: ≧90% percentile: 256 μg/l≦YKL-40
The events studied are: FIG. 5A) Unstable angina pectoris, FIG. 5B) MI, FIG. 5C) Cardiovascular death, and FIG. 5D) All-cause mortality.

FIGS. 6A and B Dipstick embodiments seen from above.
Dipstick support material (1.) with assay field (2.) for use with the biological sample and one control or standard field (3. in FIG. 6A.) or multiple control or standard fields (4a. to 4.e. in FIG. 6B.). Standards of a single (for 3.) or various (one concentration for each field in increasing or decreasing order, e.g.) YKL-40 concentrations may be applied to the control or standard fields to enable reading a positive/negative result with the stick portrayed in FIG. 6A. or assessing an approximate concentration of YKL-40 in the biological sample compared to which of the control fields in FIG. 6B. The sample/assay field resembles the most post testing.

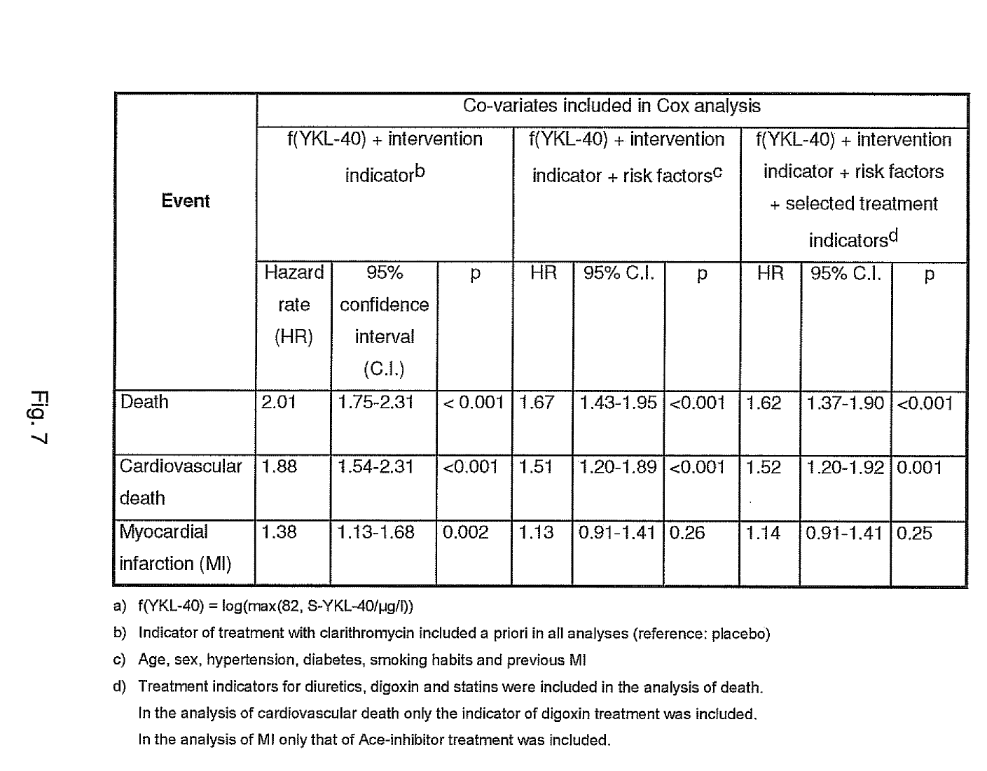

FIG. 7 The effect of f(YKL-40) on time to death, to cardiovascular death and MI alone or in combination with risk factors and risk factors plus selected indicators of treatment. Since there was no relationship between serum YKL-40 and the log of the HR of any of the events at serum YKL-40 levels below 82 μg/l and there was a linear relationship between the log of YKL40 and log HR above this value we transformed serum YKL-40 using the transformation: Y=log(max(82, serum YKL-40/μg/l)).

### EXAMPLES

Examples illustrating the correlation of YKL-40 levels to individual survival, progress of treatment of cardiovascular diseases and disorders and monitoring of individuals suffering from said diseases are provided below. These examples should not, however, be considered to limit the scope of the invention, which is defined by the appended claims.

In the examples, the abbreviation “AP” refers to angina pectoris“, “HR” refers to hazard ratio, “Ml” refers to myocardial infarction, “AMI” refers to acute myocardial infarction, “min.” refers to minutes, “hrs” and “h” refer to hours, and measurement units (such as “ml”) are referred to by standard abbreviations.

### Example 1

Patients

The patients were included in the previous published randomized, placebo controlled, multicentre CLARICOR trial of patients with stable coronary artery disease treated for two weeks with oral clarithromycin 500 mg once daily (Klacid Uno®, Abbott, UK) or a matching placebo (Jespersen et al., 2006). The patients (aged 18 to 85 years) had a diagnosis of myocardial infarction or angina pectoris (ICD codes 209-219) during the years 1993 to 1999 and were alive in August 1999. 4373 patients were eligible for participation in the study and randomized between Oct. 5, 1999 and Apr. 15, 2000. The patients were eligible if they had a history of myocardial infarction, angina, percutaneous transluminal coronary angioplasty, or coronary bypass surgery as described previously (Jespersen et al., 2006). 4350 patients gave blood, and serum was available for YKL-40 determination in 4298 of the patients. Patients completed at randomization an electronic record form with information about previous myocardial infarction, angina pectoris, percutaneous coronary intervention, coronary bypass surgery, arterial hypertension, diabetes mellitus, smoking, and medical treatment.

Follow-Up

No patient follow-up visits were planned. Information about death came from the Danish Central Civil Register, which records the vital status of all inhabitants. Information about fatal and non-fatal admissions came from the Danish National Hospital Register, a database of all somatic hospital admissions. Registration is 100% in these registers. On the basis of these registers, the coordinating centre collected death certificates and copies of hospital records and forwarded each potential event separately to the event committee during the initial 2.6 years follow-up period and all the collected death certificates for the remaining 6 years follow-up period as described elsewhere (Jespersen et al., 2006, Gluud et al., 2007).

Endpoints

In the present biomarker study we examined from randomization to re-admission for I. death, cardiaovascular death, non-fatal MI, unstable angina pectoris, II. non-fatal MI or unstable angina pectoris, and III. time to non-fatal MI, unstable angina pectoris or death. An elevation of cardiac enzymes (creatine kinase-isoenzyme MB or troponin) and significant ST changes in the electrocardiograph consistent with myocardial ischemia or myocardial infarction were required for the diagnoses of myocardial infarction. Long lasting chest pain or chest pain at rest without major changes in enzymes was classified as unstable angina.

Ethics

The study was approved by the local ethics committee (KF 01-076/99, the Danish Medicines Agency (2612-975), and the Danish Data Protection Agency (199-1200-174), and was conducted according to the Declaration of Helsinki. Participants gave written informed consent.

YKL-40 Analysis

Serum concentrations of YKL-40 were determined in duplicates by a commercial two-site, sandwich-type type enzyme-linked immunosorbent assay (ELISA) (Quidel Corporation, San Diego, Calif.) (Harvey 1998) using streptavidin-coated microplate wells, a biotinylated-Fab monoclonal capture antibody, and an alkaline phosphatase-labeled polyclonal detection antibody. The recovery of the ELISA is 102% (Harvey et al.,1998; and personal observation). The detection limit is 20 μg/L (Harvey et al, 1998). The intra-assay CVs are 5.0% (mean YKL-40 concentration 40 μg/L, n=40), 3.9% (mean 104 μg/L, n=40) and 3.8% (mean 155 μg/L, n=40). The inter-assay CVs are 5.3% (mean 42 μg/L, n=277) and 6.3% (mean 151 μg/L, n=277 (personal observation)).

Statistical Analysis

The distributions of the time to an event were calculated using Kaplan-Meier's method and compared for significant differences using Breslow's test. Cox-analyses were used to analyze the effect of one or more covariates on the time to an event. Extended Cox analyses including a covariate, time, and the interaction between time and the covariate as variables was used to test the proportionality assumption of the Cox model. The latter test was supplemented by a visual assessment of the log—log plots of the groups of the covariate. Since the distributions of serum YKL-40 as well as those of log (YKL-40) differed significantly from the Gaussian distribution as assessed by the Shapiro Wilk W test non-parametric tests (Mann-Whitney test for two groups and Kruskal Wallis test for more than two groups) were used to compare serum YKL-40 levels between patient groups. The analyses were done using the SPSS version 15.0.

To examine if the time to a specified event was significantly related to serum YKL-40 of the patients the serum concentrations of YKL-40 were transformed into a categorical variable using the 10 percentiles of the serum YKL-40 distribution as cut off limits. The Kaplan-Meier survival curves of the 10 groups were compared for significant differences. The intervention indicator was included as a stratifying variable to compensate for the effect of clarithromycin on cardiovascular death (19). If the conclusions were the same the analysis was pooled over the intervention group strata. It was finally assessed if the relationship between YKL-40 group and mean survival time was a monotonically decreasing function.

The assumption of the Cox model of linearity between serum YKL-40 and the logarithm of the hazard ratio (HR) was assessed from an inspection of the relationship between the log HR and the serum YKL-40 mean value of the serum YKL-40 percentile groups. The first group was used as the reference for the HR.

In an additional analysis, there was found no relationship between the serum-YKL-40 level and the log of the hazard rate (HR) of any of the events at serum-YKL-40 values below 82 μg/l, and there was found a linear relationship between the log of YKL-40 and log HR above this value; we transformed YKL-40 using the transformation: Y (the transformed serum YKL-40)=log(max(82, Serum-YKL-40/μg/l)). Age fulfilled the linearity assumption in case of the events all cause death and cardiac death. For MI the transformation Y=max(63, age/year) was used. If transformed YKL-40 had a significant effect on the time to an event we repeated the analysis adjusting for the known risk factors (age, sex, previous MI, smoking status, hypertension, diabetes mellitus). If the effect was still significant we included selected indicators of treatment in the analysis. These factors were selected as follows: A random sample comprising one half of the patients was selected and an analysis of the corresponding data material including as co-variates the risk factors and all indicators of medical treatment (betablocker, Ace-inhibitor, calcium blocker, statin, magnyl, long-lasting nitrate, digoxin, diuretica, anti-arrhytmica) was done. In this analysis all covariates except the treatment indicators were forced to stay in the analysis which comprised a backward elimination using the likelihood ratio test with p=0.10 for removal of variables. The analysis was repeated using the other half of the data material. The treatment indicators selected were those that were retained in both of these two analyses.

The level of significance used was 0.05 and all tests were two-sided. To account for the inflation of the experiment wise Type I error due to multiple testing we used the Bonferroni correction giving a significance level of 0.05/22=0.0023 since 22 tests were done. The analyses were done using the SPSS version 15.0.

Results

Serum YKL-40 in Relation to Demographic and Clinical Characteristics of the Subjects.

The demographic data of the included patients with stable coronary artery disease are described in FIG. 1. The mean age at entry was 65 years (Range: 30 to 85 years). A total of 31% of the 4298 enrolled patients were women and 68% had had a previous acute myocardial infarction. The median serum YKL-40 in the 4298 patients was 110 μg/l (range 20 to 3047 μg/l). No difference was found in serum YKL-40 between patients enrolled to treatment with clarythromycin or placebo. Serum YKL-40 was not significantly different in patients with and without a previous MI (median 111 μg/l, range 20 to 3047 μg/l compared to 106 μg/l, range 20 to 2802 μg/l).

Since the hazard ratio (HR) between each of the second to fifth percentile did not differ significantly from the lowest percentile I at the 1% level of significance for any of the events the first five percentiles were combined into one to obtain 6 YKL-40 groups (see text to FIG. 1). The analyses within the YKL-40 groups demonstrated, that increasing serum YKL-40 was associated with increasing age (P<0.0005), hypertension (P<0.0005), and diabetes mellitus (P<0.0005) (FIG. 1), but not with sex, previous MI, or smoking at entry.

Serum YKL-40 as a Risk Factor of Overall Death, Cardiovascular Death, Myocardial Infarction (Ml) and Unstable Angina Pectoris (UAP)

During the 2.6 years follow-up a new cardiac event occurred in 330 patients (7.6%), including 115 patients with unstable AP (2.6%) and 219 with MI (5.0%).

When comparing the patients with serum YKL-40 below 110 μg/l to those above, the higher group was not significantly associated with the occurrence of unstable AP, but significantly associated with MI (FIG. 4, P=0.002) during the 2.6 years follow-up. An analysis performed subsequent to the one above took into account cardiac events that were registered off hospital grounds. The recalculation gave rise to the following numbers for the 2.6 years follow-up: a new cardiac event occurred in a total of 390 patients (9.1%), including 120 patients with unstable AP (2.8%) and 270 with MI (6.3%), 17 patients were registered with both a UAP and MI (0.4%) (FIGS. 2 and 3). A total of 187 patients suffered cardiovascular death (4.3%) and totally 377 patients died (8.7%).

The proportionality assumption of the Cox model was fulfilled. The assumption of the Cox model of linearity between serum YKL-40 and the logarithm of the HR was not fulfilled in any of the analyses. A Cox analysis including the YKL-40 categories and the intervention indicator showed (see FIGS. 2 and 3) that serum YKL-40 significantly predicted MI (0.004), cardiovascular death (p<0.0005) as well as all-cause mortality (p<0.0005), but not to unstable AP during the 2.6 years follow-up. Multivariate adjustment for cardiovascular risk factors (<60 compared to ≧60 years, sex, prior MI, smoking status, hypertension, and diabetes mellitus) only altered the results slightly (FIG. 3).

The HRs for all-cause death increased significantly with increasing serum YKL-40 and they were in Group II 1.45, Group III 1.89, Group IV 2.37, Group V 2.59, and Group VI 3.75 compared to serum YKL-40 below 110 μg/l (Group I) (P<0.0005 for all except Group II) (FIG. 2). The HRs were only reduced slightly by the multivariate adjustment for cardiovascular risk factors (FIG. 3).

The event free survival for unstable AP, MI, cardiovascular death, and all-cause mortality during the 2.6 years of follow-up for each of the six serum YKL-40 groups is outlined in FIG. 5A-D. The incidence of unstable AP with time was independent of serum YKL-40 in the present study (FIG. 5A). However, in case of MI group I was clearly separated from the remaining groups. There was a clear reduction in event-free survival for both MI and cardiovascular death with increasing serum YKL-40. The event-free survival for MI was reduced with increasing serum YKL-40, and the proportion with MI was between 7.0% and 9.0% for the highest serum YKL-40 (Group II to VI) and 5.0% for the low serum YKL-40 (Group I) (FIG. 1B). The cardiovascular mortality was 8.0% for the highest serum YKL-40 (Group VI) and 2.6% for low serum YKL-40 (Group I) (FIG. 5C). This association was even clearer for all-cause mortality (FIG. 5D). 18.4% of the patients with the highest serum YKL-40 died within 2.6 years compared to 5.3% of the patients with low serum YKL-40. The HRs for MI, cardiovascular death and all-cause mortality were only reduced slightly by the multivariate adjustment for cardiovascular risk factors (FIG. 3). An exploratory Cox analysis showed that when patients suffering a cardiac death were excluded there was still a highly significant (p<0.0005) association between time to death and YKL-40. The same was true (p<0.0005) when patients dying from other causes than cardiac ones were excluded.

In the analysis with the cut-of point of serum-YKL-40 values at 82 μg/l, the Y (the transformed serum-YKL-40) was significantly associated with cardiovascular death (HR=1.88, 95% confidence interval (C.I.)=1.54-2.31, p<0.001), all-cause mortality (HR=2.01, 95% C.I.=1.75-2.31, p<0.001), and MI (HR=1.38, 95% C.I.=1.13-1.68, p=0.002), but not unstable angina pectoris (p=0.85) (FIG. 7). Following multivariable adjustment for cardiovascular risk factors (age, sex, previous MI, smoking status, hypertension, diabetes mellitus) Y contributed significantly to prediction of all cause mortality (HR=1.67, 95% C.I.=1.43-1.95, p<0.001) and cardiovascular mortality (HR=1.51 95% C.I.=1.20-1.89, p=0.001), but not MI (p=0.26). In the analysis where the predictive significance of medical treatments was assesses while all risk factors were forced to remain in the analysis (see section on statistical analysis) treatment with diuretics, digoxin, and statin were retained in the analysis of time to death. In the analysis of time to cardiovascular death only treatment with digoxin was retained. Following multivariable adjustment for cardiovascular risk factors and the selected medical treatment indicators Y contributed significantly to prediction of all cause mortality (HR=1.62, 95% C.I.=1.37-1.90, p<0.001) and cardiovascular mortality (HR=1.52 95% C.I.=1.20-1.92, p=0.001).

In conclusion, serum YKL-40 level has been found to be a very strong biomarker of all death, cardiac death and myocardial infarction in patients with stable coronary artery disease. Due to the low number of individuals with events of unstable angina pectoris during the follow-up period, the results where inconclusive with regard to unstable angina pectoris and YKL-40. However, the results show that YKL-40 is a surprisingly apt biomarker for monitoring the sufficiency of medical treatment in individuals with e.g. stable coronary artery disease or any other heart disease or disorder, especially those caused by atherosclerosis, and/or for classifying individuals according to a prognosis of their survival, according to a prognosis of suffering cardiovascular death, or a prognosis of the risk of suffering a myocardial infarction. Such monitoring or classification can be done by the method herein disclosed: thus by measuring the level of YKL-40 in a biological sample from said individual(s), and comparing the measured level to a reference level of YKL-40. By this method, especially serum YKL-40 levels, but also YKL-40 levels obtained from other biological samples can be of assistance in reducing the high death rate in individuals suffering from coronary artery disease or other heart diseases.

Discussion

The present study demonstrated that high serum YKL-40 concentration is a significant predictor for MI, cardiovascular death, and all-cause mortality in patients with stable CAD. There was a more than three-fold increased hazard ration in patients with the highest YKL-40 levels. In addition high serum YKL-40 was associated with increasing age, hypertension, and diabetes mellitus, but not with sex, previous MI, or smoking at entry. Correcting the analyses for these variables had no major impact on the HRs for MI, cardiovascular- and all-cause mortality.

Inflammation plays an important role in atherogenesis and atherothrombotic events and is associated with the development of myocardial infarction, stroke and cardiovascular mortality (Jialal et al., 2003; Lindahl et al., 2000; Mueller et al., 2002; Ridker et al., 2001; Ridker et al., 2002; Albert et al., 2002). YKL-40 is a new biomarker of acute and chronic inflammation in patients with stable coronary artery disease. This is supported by the finding that YKL-40 is produced by macrophages and neutrophils locally in tissues with inflammation (Johansen et al., 2006; Volck et al., 1998; Boot et al., 1999; Johansen et al., 1999; Johansen et al., 2005; Johansen et al., 2000). YKL-40 act as a growth factor for fibroblasts and chondrocytes in synergy with the protein insulin-like growth factor-1, and it is limiting the catabolic effects of tumour necrosis factor-alpha and interleukin-1 (Recklies et al., 2002; Ling et al., 2004) and IL-6 (personal observation).

### REFERENCES

Albert C M, Ma J, Rifai N, Stampfer M J, Ridker P M. Prospective study of C-reactive protein, homocysteine, and plasma lipid levels as predictors of sudden cardiac death. Circulation. 2002; 105:2595-9.

Boot R G, van Achterberg T A E, van Aken B E, Renkema G H, Jacobs M J H M, Aerts J M F G, et al. Strong induction of members of the chitinase family of proteins in atherosclerosis. Chitotriosidase and human cartilage gp-39 expressed in lesion macrophages. Arterioscler Thromb Vasc Biol 1999; 19:687-94.

Bunn, et al., U.S. Pat. No. 5,213,961

Coligan, et al., Unit 9, Current Protocols in Immunology, Wiley Interscience, 1991).

De Ceuninck F, Gaufillier S, Bonnaud A, Sabatini M, Lesur, C, Pastoureau P. YKL-40 (Cartilage gp-39) induces proliferative events in cultured chondrocytes and synoviocytes and increases glycosaminoglycan synthesis in chondrocytes. Biochem Biophys Res Commun 2001; 285:926-31.

Fiore C E, Tamborino C. YKL-40 and graft rejection. Am J Med 2000; 108:688-9.

Gluud C, Als-Nielsen B, Damgaard M, et al. Clarithromycin for two weeks for stable coronary heart disease: six-year follow-up of the CLARICOR randomized trial and updated meta-analysis of antibiotics for coronary heart disease. Resubmitted 2007.

Harlow and Lane, Antibodies, A Laboratory Manual, Cold Spring Harbor Laboratory, 1988.

Harvey S, Weisman M, O'Dell J, et al. Chondrex: new marker of joint disease. Clin Chem 1998; 44:509-16.

Innis, et al., PCR Protocols: A Guide to Methods and Applications, Acad. Press, 1990.

Jespersen C M, Als-Nielsen B, Damgaard M, et al. Randomised placebo controlled multicentre trial to assess short term clarithromycin for patients with stable coronary heart disease: CLARICOR trial. BMJ 2006; 332:22-7

Jialal I, Devaraj S. Role of C-reactive protein in the assessment of cardiovascular risk. Am J Cardiol 2003; 91:200-2.

Johansen J S, Baslund B, Garbarsch C, Garbarsch C, Hansen M, Stoltenberg M, et al. YKL-40 in giant cells and macrophages from patients with giant cell arteritis. Artritis Rheum 1999; 42:2624-30.

Johansen J S, Christoffersen P, Møller S, Price P A, Henriksen J H, Garbarsch C, et al. Serum YKL-40 is increased in patients with hepatic fibrosis. J Hepatol 2000; 32:911-20.

Johansen J S, Milman N, Hansen M, Garbarsch C, Price P A, Graudal N. Increased serum YKL-40 in patients with pulmonary sarcoidosis. A potential marker of disease activity? Respiratory Medicine 2005; 99:396-402.

Johansen J S, Jensen B V, Roslind A, Nielsen D, Price P A. Review. Serum YKL-40, a new prognostic biomarker in cancer patients? Review. Cancer Epidemiol Biomarkers Prev 2006; 15:194-202.

Junker N, Johansen J S, Hansen L T, Lund E L, Kristjansen P G. Regulation of YKL-40 expression during genotoxic or microenvironmental stress in human glioblastoma cells. Cancer Sci 2005; 96:183-90.

Kamal S M, Turner B, He Q, Rasenack J, Bianchi L, Al Tawil A, et al. Progression of fibrosis in hepatitis C with and without schistosomiasis: correlation with serum markers of fibrosis. Hepatology 2006; 43:771-9.

Kohler and Milstein, Nature, 256:495, 1975

Kohsaka, et al., Nuc. Acids Res., 21:3469-3472, 1993

Langone, et al. eds. (Acad. Press, 1981).

Lindahl B, Toss H, Siegbahn A, Venge P, Wallentin L. Markers of myocardial damage and inflammation in relation to long-term mortality in unstable coronary artery disease. N Engl J Med 2000; 343:1139-47.

Ling H, Recklies A D. The chitinase 3-like protein human cartilage glycoprotein 39 inhibits cellular responses to the inflammatory cytokines interleukin-1 and tumour necrosis factor-alpha. Biochem J 2004; 380:651-9.

Millis A J T, Hoyle M, Kent L. In vitro expression of a 38,000 dalton heparin-binding glycoprotein by morphologically differentiated smooth muscle cells. J Cell Physiol 1986; 127:366-72.

Mueller C, Buettner H J, Hodgson J M, Marsch S, Perruchoud A P, Roskamm H, Neumann F J. Inflammation and long-term mortality after non-ST-elevation acute coronary syndrome treated with a very early invasive strategy in 1042 consecutive patients. Circulation 2002; 105:1412-5.

Nishikawa K C, Millis A J T. gp38k (CHI3L1) is a novel adhesion and migration factor for vascular cells. Exp Cell Res 2003; 287:79-87.

Nøjgaard C, Johansen J S, Christensen E, Skovgaard L T, Price P A, Becker U, et al. Serum levels of YKL-40 and PIIINP as prognostic markers in patients with alcoholic liver disease. J Hepatol 2003; 39:179-86.

Recklies A D, White C, Ling H. The chitinase 3-like protein human cartilage 39 (HC-gp39) stimulates proliferation of human connective-tissue cells and activates both extracellular signal-regulated kinase-and protein kinase B-mediated signalling pathways. Biochem J 2002; 365:119-26

Recklies A D, Ling H, White C, Bernier S M. Inflammatory cytokines induce production of CHI3L1 by articular chondrocytes. J Biol Chem. 2005; 280:41213-21.

Remington's Pharmaceutical Sciences, 16th Ed., Mack Publishing Co., Easton, Pa., 1980.

Renkema G H, Boot R G, Au F L, et al. Chitotriosidase, a chitinase, and the 39-kDa human cartilage glycoprotein, a chitin-binding lectin, are homologues of family 18 glycosyl hydrolases secreted by human macrophages. Eur J Biochem 1998; 251:504-9.

Ridker P M, Stampfer M J, Rifai N. Novel risk factors for systemic atherosclerosis:a comparison of C-reactive protein, fibrinogen, homocysteine, lipoprotein(a), and standard cholesterol screening as predictors of peripheral arterial disease. JAMA 2001; 285:2481-5.

Ridker P M, Rifai N, Rose L, Buring J E, Cook N R. Comparison of C-reactive protein and low-density lipoprotein cholesterol levels in the prediction of first cardiovascular events. N Engl J Med 2002; 347:1557-65.

Salacinski, et al., Anal. Biochem., 117:136-146, 1981

Shackelton L M, Mann D M, Millis A J T. Identification of a 38-kDa heparin-binding glycoprotein (gp38k) in differentiating vascular smooth muscle cells as a member of a group of proteins associated with tissue remodelling. J Biol Chem 1995; 270:13076-83.

Vind I, Johansen J S, Price P A, Munkholm P. Serum YKL-40, a potential new marker of disease activity in patients with inflammatory bowel disease. Scand J Gastroenterol 2003; 38:599-605.

Volck B, Price P A, Johansen J S, Sørensen O, Benfield T, Calafat J, et al. YKL-40, a mammalian member of the chitinase family, is a matrix protein of specific granules in human neutrophils. Proc Assoc Am Phys 1998; 110:351-60.

1. A method for classifying individuals suffering from heart disease caused by atherosclerosis according to a prognosis of their survival, said method comprising: measuring the level of YKL-40 in a biological sample from said individual, and comparing the measured level to a reference level of YKL-40.

2. A method for monitoring the health state of an individual suffering from heart disease caused by atherosclerosis in relation to a prognosis of their survival, said method comprising: measuring the level of YKL-40 in a biological sample from said individual; and comparing the measured level to a reference level of YKL-40.

3. The method according to claim 1, wherein the individual suffers from atherosclerotic coronary artery disease.

4. The method according to claim 1, wherein the heart disease is a stable coronary artery disease.

5. The method according to claim 1, wherein the measured level of YKL-40 is a level above about 80 μg/l.

6. The method according claim 1, wherein the reference level is a cut-off value of about 80 μg/l.

7. The method according to claim 1, wherein the reference level of YKL-40 is a set of cut-off values.

8. The method according to claim 7, wherein the set of cut-off values is selected from one or more of the following cut-off values: about 80 μg/l, about 90 μg/l, about 100 μg/l, about 110 μg/l, about 120 μg/l, about 130 μg/l, about 140 μg/l, about 150 μg/l, about 160 μg/l, about 170 μg/l, about 180 μg/l, about 190 μg/l, about 200 μg/l, about 210 μg/l, and about 220 μg/l.

9. The method according to claim 1, wherein the prognosis of their survival is a prognosis of the risk of suffering a myocardial infarction.

10-15. (canceled)

16. The method according to claim 1, wherein the YKL-40 level is measured together with the level of another biomarker.

17. The method according to claim 16, wherein the other biomarker is selected from the group consisting of C-reactive protein (CRP), brain natriuretic protein (BNP), interleukins, tumor necrosis factor-alfa, homocysteine, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Myosin light chain-1 (MLC-1), P-selectin and CKMB.

18. The method according to claim 1, wherein the YKL-40 level is measured by use of a dipstick.

19. The method according to claim 1, wherein the biological sample is blood, serum, or plasma.

20. The method according to claim 1, wherein the individual is a human.

21. A kit of parts comprising means for detecting YKL-40 in a biological sample; means for comparing the measured level of YKL-40 with a reference level of YKL-40; and instruction on how to classify and/or monitor individuals suffering from heart disease caused by atherosclerosis according to a prognosis of their survival according to their YKL-40 levels, when the YKL-40 levels are above 80 μg/l.

22. The kit of parts according to claim 21, wherein the kit further comprises means of detecting additional biomarkers; preferably additional biomarkers selected from the group consisting of C-reactive protein, homocysteine, brain natriuretic protein, interleukins, tumor necrosis factor-alfa, homocystein, amyloid A protein, Pregnancy-Associated Plasma Protein-A, troponines, soluble intercellular adhesion molecule-1, soluble UPAR, the aminoterminal propeptide of type III procollagen (P-III-NP), monocyte chemoattractant protein-1, fibrin D-dimer, Growth-differentiation factor-15, Ischemia-modified albumin, lipoprotein-associated phospholipase A2, matrix metalloproteinases and CKMB; more preferably additional biomarkers selected from the group consisting of C-reactive protein, brain natriuretic protein and/or homocysteine.

23. The kit of parts according to claim 21, wherein the means for detecting YKL-40 in a biological sample; and means for comparing the measured level of YKL-40 with at least one reference level of YKL-40 are comprised in at least one device.

24. The kit of parts according to claim 23, wherein the device is a dipstick.

25. The kit of parts according to claim 23, wherein the device comprises a single reference level, representing a cut-off value.

26. The kit of parts according to claim 23, wherein the device comprises means for comparing the measured level of YKL-40 with a set of cut-off values for YKL-40.
